(19) United States
(12) Patent Application Publication
(10) Pub. No.: US 2022/0062563 A1
(43) Pub. Date: Mar. 3, 2022

(54) HYPODERMIC INTERFACE ASSEMBLY

(71) Applicant: Neogen Corporation, Lansing, MI (US)

(72) Inventors: Gregory S. Hastings, Richmond, KY (US); Bradley E. Galbreath, Lexington, KY (US); Nicholas J. Wagner, Lexington, KY (US); Taylor Kopacka Leigh, Alpharetta, GA (US); Peter Wyndham Shipp, JR., Woodstock, GA (US); Jason Lye, Atlanta, GA (US); Randall M. Bachtel, Lawrenceville, GA (US); Dexter E. Jacobs, Austin, TX (US); Michael Trovato, Portland, OR (US)

(21) Appl. No.: 17/008,936

(22) Filed: Sep. 1, 2020

(51) Int. Cl. A61M 5/34; A61M 5/32

(52) U.S. Cl. CPC A61M 5/343; A61M 5/346; A61M 5/3293

(57) ABSTRACT

A hypodermic interface assembly includes a hub, a cannula carrier connected to the hub, and a cannula connected to the cannula carrier. The cannula carrier includes an outer side surface including a first outer side surface portion, a second outer side surface portion, and a third outer side surface portion. The cannula extends through a passage formed by the cannula carrier.

### TECHNICAL FIELD

The disclosure relates generally to hypodermic interface assemblies.

### BACKGROUND

This section provides background information related to the present disclosure and is not necessarily prior art.

While known hypodermic interface assemblies have proven to be acceptable for various applications, such hypodermic interface assemblies are nevertheless susceptible to improvements that may enhance their overall performance and cost. Therefore, a need exists to develop hypodermic interface assemblies that advance the art.

### SUMMARY

This section provides a general summary of the disclosure, and is not a comprehensive disclosure of its full scope or all of its features.

In one aspect, the invention is a hypodermic interface assembly including: (a) a hub having a proximal end having a proximal end surface, a distal end having a distal end surface, an outer side surface extending between and connecting the proximal end surface to the distal end surface, and an inner surface extending between and connecting the proximal end surface to the distal end surface, wherein the inner surface defines a passage extending through the hub from the proximal end surface to the distal end surface; (b) a cannula carrier friction-fit connected to the hub, wherein the cannula carrier includes an outer side surface including a first outer side surface portion, a second outer side surface portion, and a third outer side surface portion; and (c) a cannula connected to the cannula carrier, wherein the cannula extends through a passage formed by the cannula carrier.

In one embodiment, the cannula carrier may also include a neck portion and a head portion extending from the neck portion, wherein the neck portion may be defined by the first outer side surface portion, and wherein the head portion may be defined by the second outer side surface portion and the third outer side surface portion. Further, the neck portion may be disposed within the passage formed by the hub and, optionally, the first outer side surface portion of the neck portion may be arranged proximate an inner surface that defines the passage extending through the hub.

In another embodiment, the third outer side surface portion of the head portion may be disposed proximate the distal end surface of the distal end of the hub. In a further embodiment, the first outer side surface portion may be defined by a neck portion geometry, wherein the second outer side surface portion may be defined by a head portion geometry that is greater than the neck portion geometry. In an even further embodiment, the third outer side surface portion may be a proximal-side-facing surface that joins the first outer side surface portion to the second outer side surface portion.

In another aspect, the invention is a hypodermic interface assembly including: (a) a hub having a proximal end having a proximal end surface, a distal end having a distal end surface, an outer side surface extending between and connecting the proximal end surface to the distal end surface, and an inner surface extending between and connecting the proximal end surface to the distal end surface, wherein the inner surface defines a passage extending through the hub from the proximal end surface to the distal end surface; (b) a cannula carrier connected to the hub, wherein the cannula carrier includes an outer side surface including a first outer side surface portion, a second outer side surface portion, and a third outer side surface portion; (c) a cannula connected to the cannula carrier, wherein the cannula extends through a passage formed by the cannula carrier; and (d) one or more fastening portions that non-removably-secures one or both of the cannula carrier to the hub and the cannula to the cannula carrier.

In other embodiments, the one or more fastening portions may include one fastening portion that non-removably-secures the cannula carrier to the hub; the one or more fastening portions may include one fastening portion that non-removably-secures the cannula to the cannula carrier; the one or more fastening portions may include a first fastening portion that non-removably-secures a first portion of the cannula to a proximal end of the cannula carrier and a second fastening portion that non-removably-secures a second portion of the cannula to a proximal end of the cannula carrier; the one or more fastening portions may include a first fastening portion that non-removably-secures the cannula carrier to the hub, a second fastening portion that non-removably-secures a first portion of the cannula to a proximal end of the cannula carrier, and a third fastening portion that non-removably-secures a second portion of the cannula to a proximal end of the cannula carrier; or the third outer side surface portion may be a proximal-side-facing surface that joins the first outer side surface portion to the second outer side surface portion.

In yet another embodiment, the cannula carrier may further include a neck portion and a head portion extending from the neck portion, wherein the neck portion is defined by the first outer side surface portion, and wherein the head portion is defined by the second outer side surface portion and the third outer side surface portion. Further, the neck portion may disposed within the passage formed by the hub. Even further, the first outer side surface portion of the neck portion may be arranged proximate an inner surface that defines the passage extending through the hub.

In one embodiment, the third outer side surface portion of the head portion may be disposed proximate the distal end surface of the distal end of the hub; or the first outer side surface portion may be defined by a neck portion geometry, wherein the second outer side surface portion is defined by a head portion geometry that is greater than the neck portion geometry.

In yet another aspect, the invention is a hypodermic interface assembly including: (a) a hub having a proximal end with a proximal end surface, a distal end with a distal end surface, an outer side surface extending between and connecting the proximal end surface to the distal end surface, and an inner surface extending between and connecting the proximal end surface to the distal end surface, wherein the inner surface defines a passage extending through the hub from the proximal end surface to the distal end surface; (b) a cannula carrier connected to the hub, wherein the cannula carrier includes an outer side surface including a first outer side surface portion, a second outer side surface portion, and a third outer side surface portion; and (c) a cannula connected to the cannula carrier, wherein the cannula extends through a passage formed by the cannula carrier, wherein the cannula carrier is formed from a material having a hardness that permits at least a portion of the cannula carrier to deform and deviate from (i) an at-rest state axis extending through the cannula carrier and then to (ii) a flexed axis extending through the portion of the cannula carrier upon application of a force imparted to the cannula that is not coaxial with the at-rest state axis extending through the cannula carrier and then back to the at-rest state axis extending through the cannula carrier, whereby a location of stress concentration applied to the cannula arising from the force imparted to the cannula is axially shifted from the hub toward a distal end of the cannula carrier for reinforcing a structural integrity of the cannula.

Another aspect of the invention is a method including providing a hub and a cannula carrier friction-fit connected to the hub, wherein the cannula carrier includes an outer side surface including a first outer side surface portion, a second outer side surface portion, and a third outer side surface portion non-separably joining a cannula to the cannula carrier, and wherein the cannula extends through a passage formed by the cannula carrier. The method also may include separably joining the hub to an injection gun and inserting the cannula into the flesh of a subject. Yet further, the method may include subjecting one or both of the cannula and the hub to one or more radial forces relative to a central axis extending through the cannula and the hub, whereby a central axis extending through the cannula carrier is permitted to deviate from the central axis extending through the hub while the hub remains separably joined to the injection gun and the cannula is removably disposed within the flesh of the subject.

The details of one or more implementations of the disclosure are set forth in the accompanying drawings and the description below. Other aspects, features, and advantages will be apparent from the description and drawings, and from the claims.

### DESCRIPTION OF DRAWINGS

The drawings described herein are for illustrative purposes only of selected configurations and not all possible implementations, and are not intended to limit the scope of the present disclosure.

FIG. 1 is an exploded perspective view of an exemplary hypodermic interface assembly.

FIG. 2 is a perspective view of an exemplary cannula of the hypodermic interface assembly of FIG. 1.

FIG. 3 is a front perspective view of an exemplary hub of the hypodermic interface assembly of FIG. 1.

FIG. 4 is a rear perspective view of the hub of FIG. 2.

FIG. 5 is another front perspective view of the hub of FIG. 2.

FIG. 6 is a side view of the hub of FIG. 2.

FIG. 7 is a cross-section view of the hub according to line 7-7 of FIG. 6.

FIG. 8A is another side view of the hub of FIG. 2.

FIG. 8B is a top view of the hub according to arrow 8B of FIG. 8A.

FIG. 9A is another side view of the hub of FIG. 2.

FIG. 9B is a top view of the hub according to arrow 9B of FIG. 9A.

FIG. 10 is a bottom view of the hub according to arrow 10 of FIG. 8A or 9A.

FIG. 11 is a front perspective view of an exemplary cannula carrier of the hypodermic interface assembly of FIG. 1.

FIG. 12 is a rear perspective view of the cannula carrier of FIG. 11.

FIG. 13 is another rear perspective view of the cannula carrier of FIG. 11.

FIG. 14 is another front perspective view of the cannula carrier of FIG. 11.

FIG. 15 is a top view of the cannula carrier according to arrow 15 of FIG. 17 of FIG. 17.

FIG. 16 is a bottom view of the cannula carrier according to arrow 16 of FIG. 17.

FIG. 17 is a side view of the cannula carrier of FIG. 11.

FIG. 18 is a cross-section view of the cannula carrier according to line 18-18 of FIG. 17.

FIG. 19A is a cross-sectional view of a partially assembled hypodermic interface assembly arranged in a first partially assembly state according to line 19-19 of FIG. 1.

FIG. 19B A is a cross-sectional view of a partially assembled hypodermic interface assembly arranged in a second partially assembly state according to line 19-19 of FIG. 1.

FIG. 19C is a cross-sectional view of a partially assembled hypodermic interface assembly arranged in a third partially assembly state according to line 19-19 of FIG. 1.

FIG. 19D is a cross-sectional view of a partially assembled hypodermic interface assembly arranged in a fourth partially assembly state according to line 19-19 of FIG. 1.

FIG. 19E is a cross-sectional view of an assembled hypodermic interface assembly according to line 19-19′ of any of FIGS. 20, 23, 24, and 25.

FIG. 19E′ is another view of the assembled hypodermic interface assembly according to FIG. 19E illustrating a portion of a length of a tube-shaped body of the cannula carrier bent, flexed, or deviated away from its central axis.

FIG. 19E″ is yet another view of the assembled hypodermic interface assembly according to FIG. 19E′ illustrating the portion of the length of the tube-shaped body of the cannula carrier bent, flexed, or deviated away from its central axis in another direction opposite the bent, flexed, or deviated direction of FIG. 19E′.

FIG. 20 is an assembled front perspective view of the hypodermic interface assembly of FIG. 1.

FIG. 21 is a top view of the hypodermic interface assembly according to arrow 21 of FIG. 20.

FIG. 22 is a bottom view of the hypodermic interface assembly according to arrow 22 of FIG. 20.

FIG. 23 is an assembled rear perspective view of the hypodermic interface assembly of FIG. 1.

FIG. 24 is another assembled front perspective view of the hypodermic interface assembly of FIG. 1.

FIG. 25 is a side view of the hypodermic interface assembly of FIG. 1.

FIG. 26 is a cross-sectional view of a partially assembled exemplary hypodermic interface assembly arranged in a first partially assembly state.

FIG. 27 is a cross-sectional view of a partially assembled exemplary hypodermic interface assembly of FIG. 26 arranged in a second partially assembly state.

FIG. 28 is a cross-sectional view of an assembled exemplary hypodermic interface assembly of FIG. 27.

FIG. 29 is a cross-sectional view of a partially assembled exemplary hypodermic interface assembly arranged in a first partially assembly state.

FIG. 30 is a cross-sectional view of a partially assembled exemplary hypodermic interface assembly of FIG. 29 arranged in a second partially assembly state.

FIG. 31 is a cross-sectional view of an assembled exemplary hypodermic interface assembly of FIG. 29.

FIG. 32 is a view of a hypodermic interface assembly arranged proximate animalia.

FIG. 33A is a side view of the hypodermic interface assembly and a cross-sectional view of a portion of the animalia of FIG. 32 arranged in a spaced-apart relationship.

FIG. 33B is another side view of the hypodermic interface assembly and another cross-sectional view of a portion of the animalia according to FIG. 33A arranged in a pierced relationship.

FIG. 33C is another side view of the hypodermic interface assembly and another cross-sectional view of a portion of the animalia according to FIG. 33B arranged in a pierced relationship while, optionally, the hypodermic interface assembly is utilized for injecting a fluid into the animalia.

FIG. 33D is another side view of the hypodermic interface assembly and another cross-sectional view of a portion of the animalia according to FIG. 33B arranged in a pierced-and-flexed relationship.

FIG. 33E is another side view of the hypodermic interface assembly and another cross-sectional view of a portion of the animalia according to FIG. 33D arranged in a flexed-after-pierced relationship.

FIG. 33F is another side view of the hypodermic interface assembly and another cross-sectional view of a portion of the animalia according to FIG. 33E arranged in a return-flexed-after-pierced relationship.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The figures illustrate exemplary implementations of hypodermic interface assemblies. Based on the foregoing, it is to be generally understood that the nomenclature used herein is simply for convenience and the terms used herein should be given the broadest meaning by one of ordinary skill in the art.

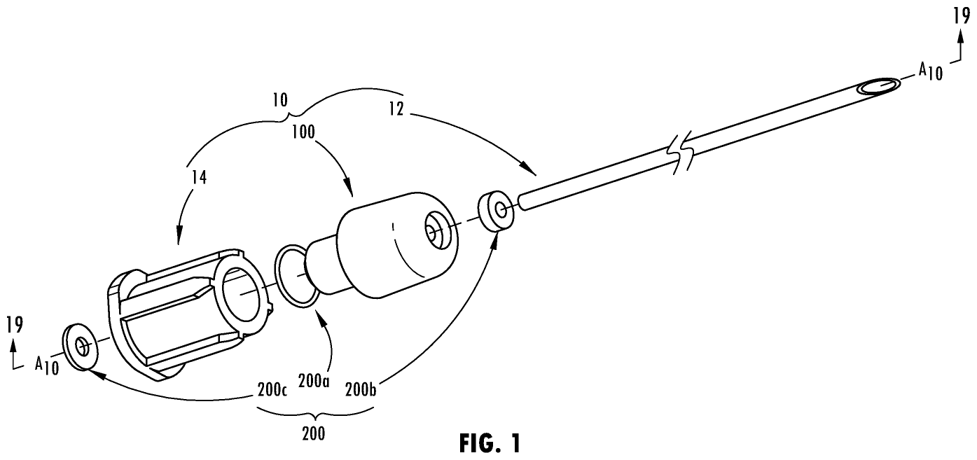

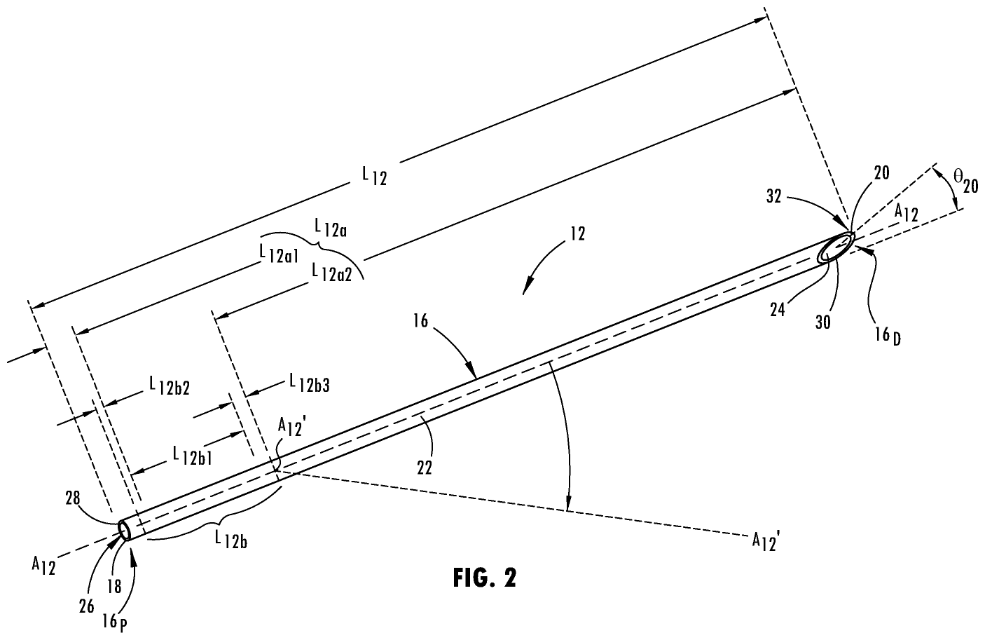

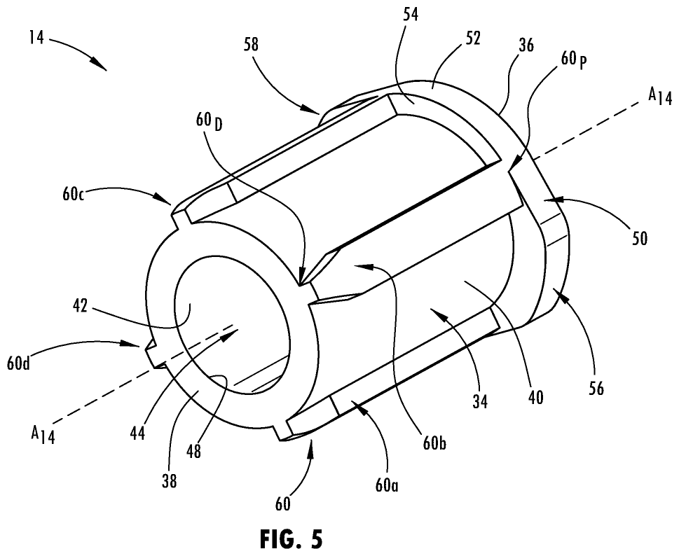

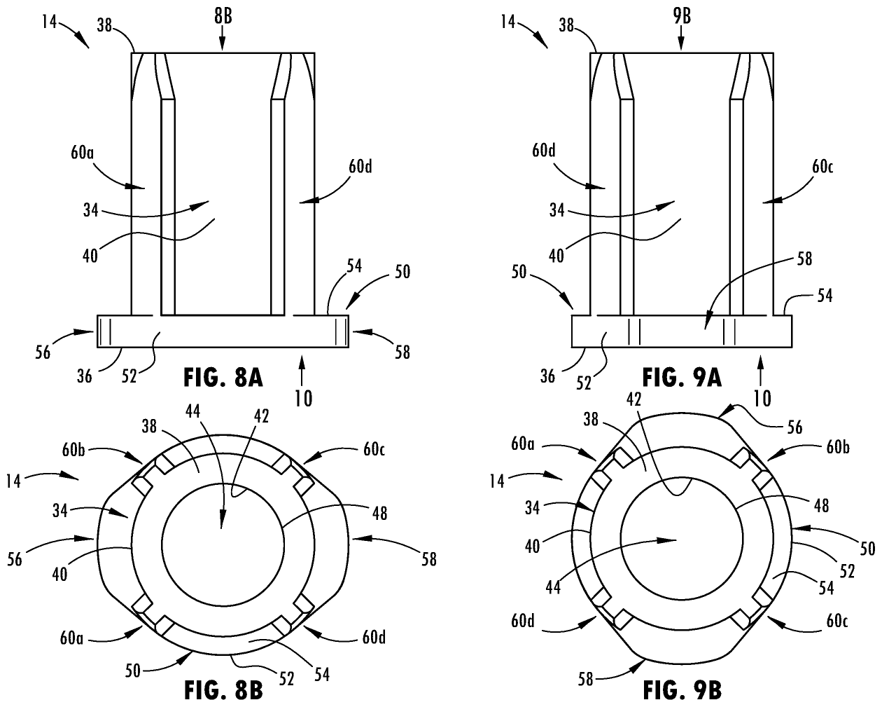

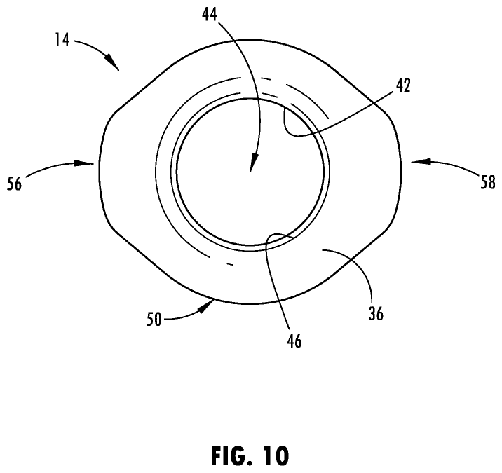

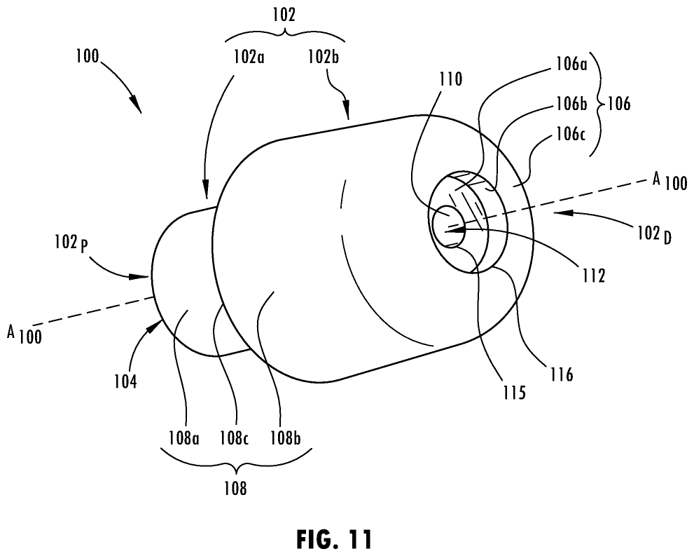

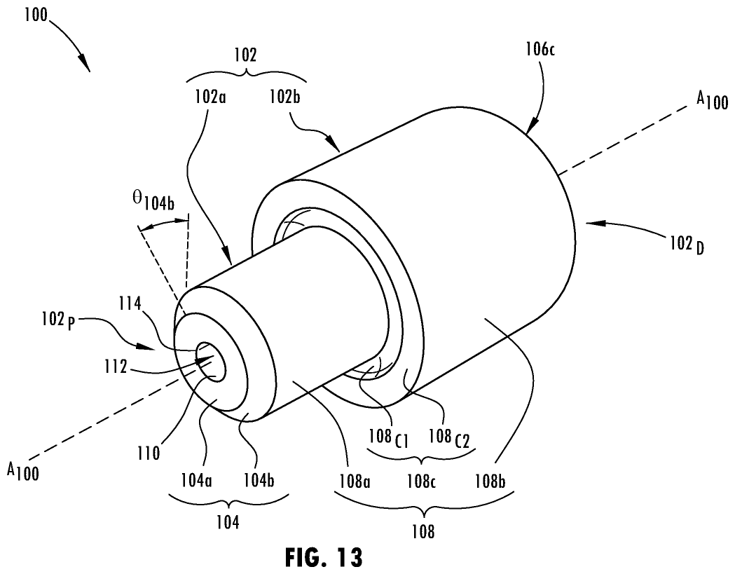

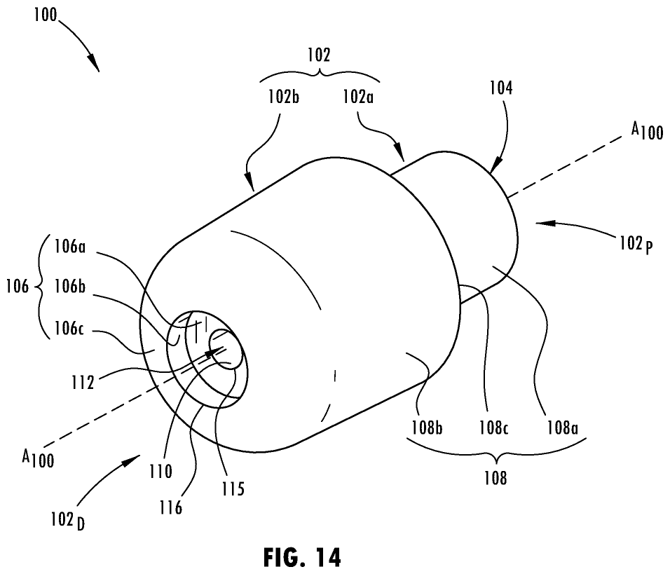

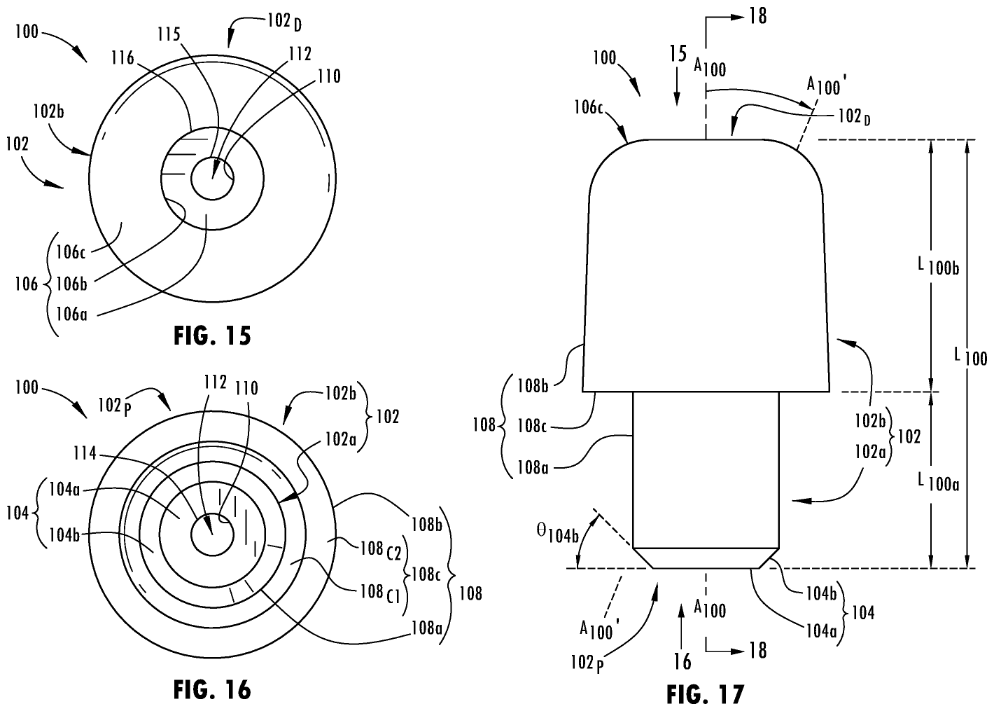

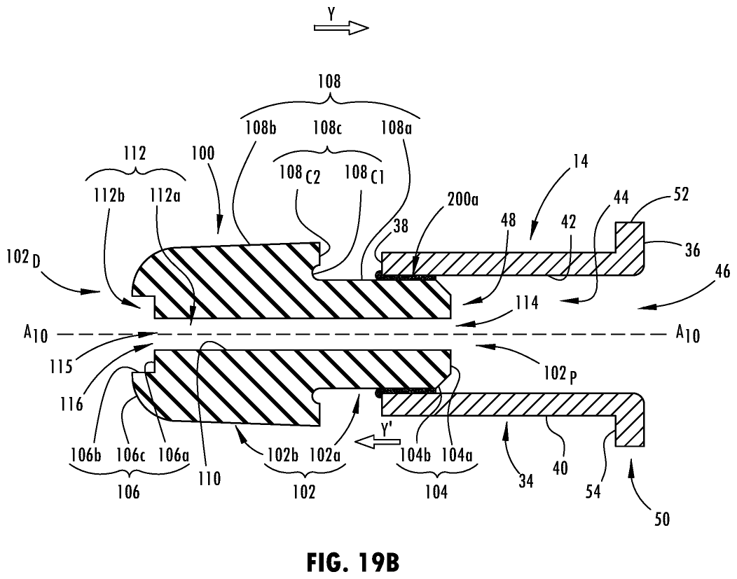

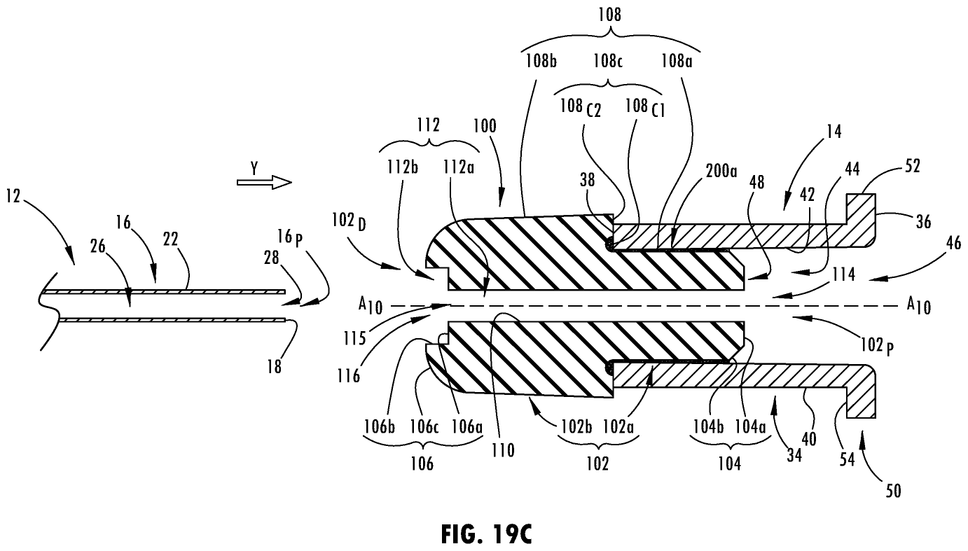

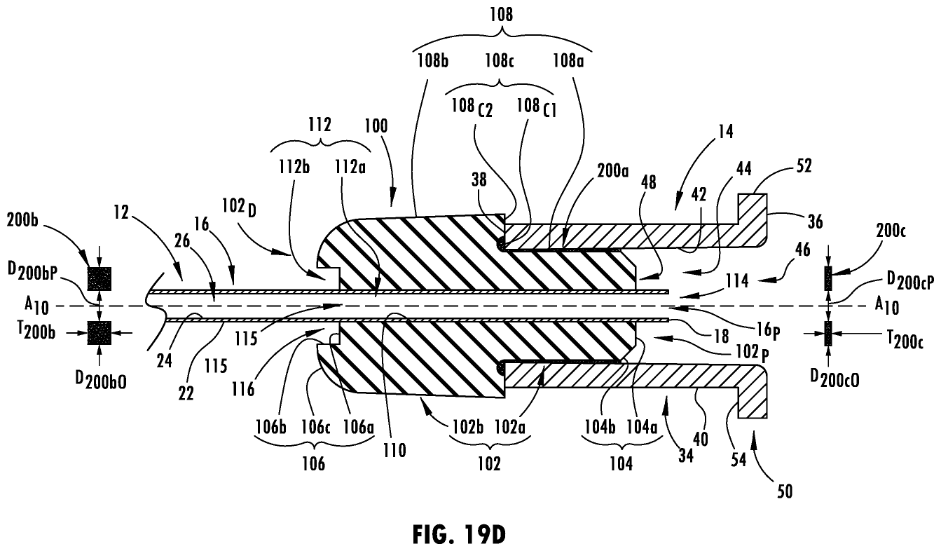

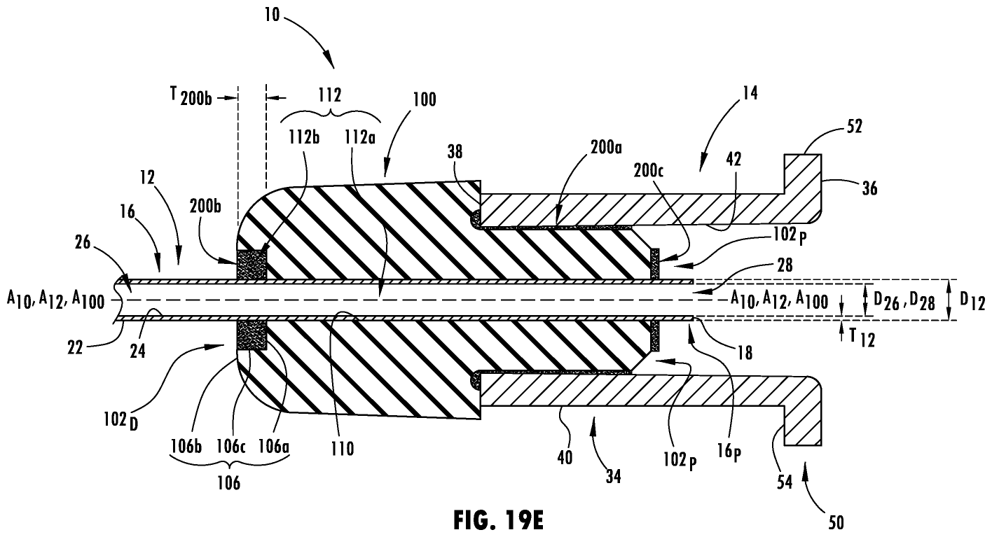

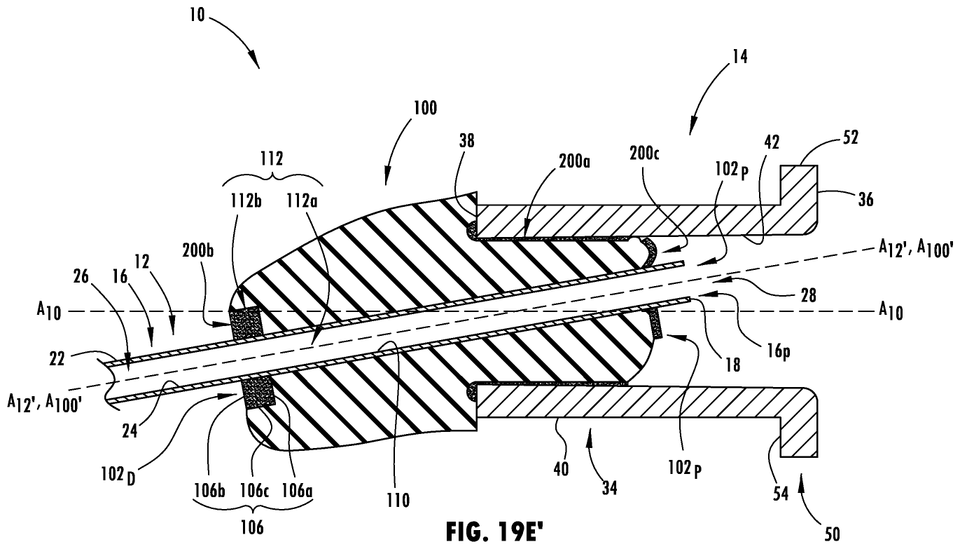

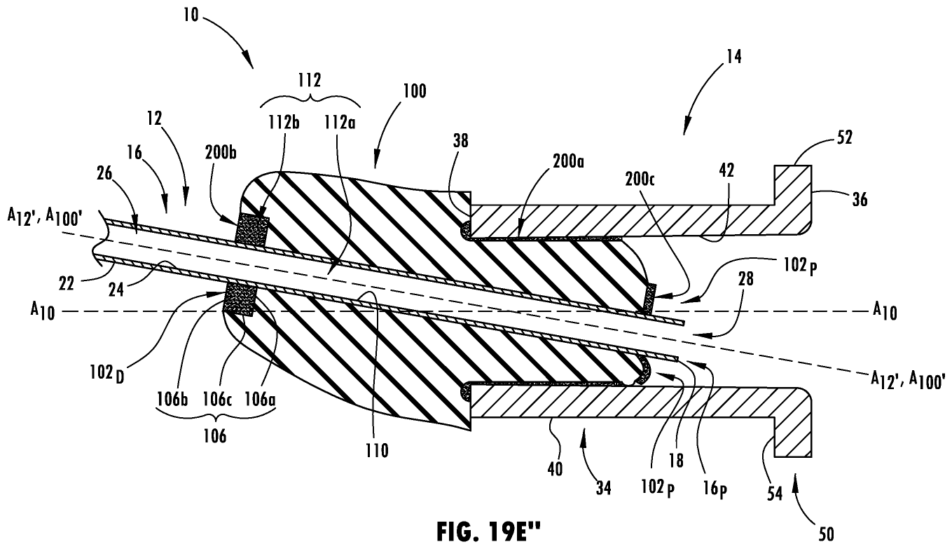

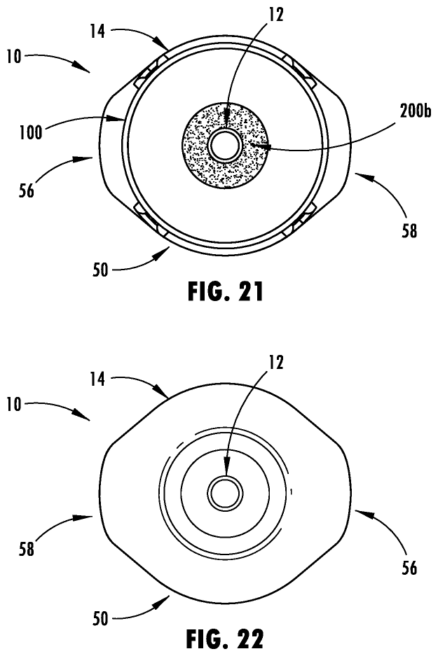

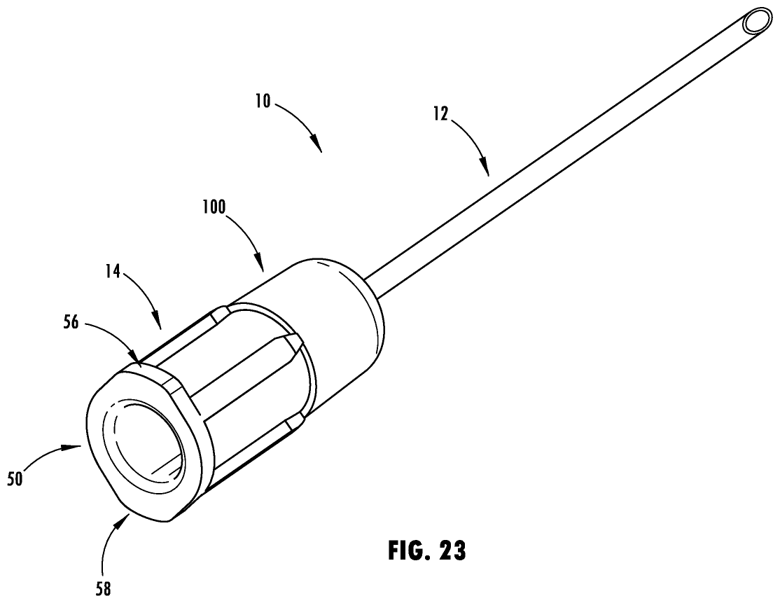

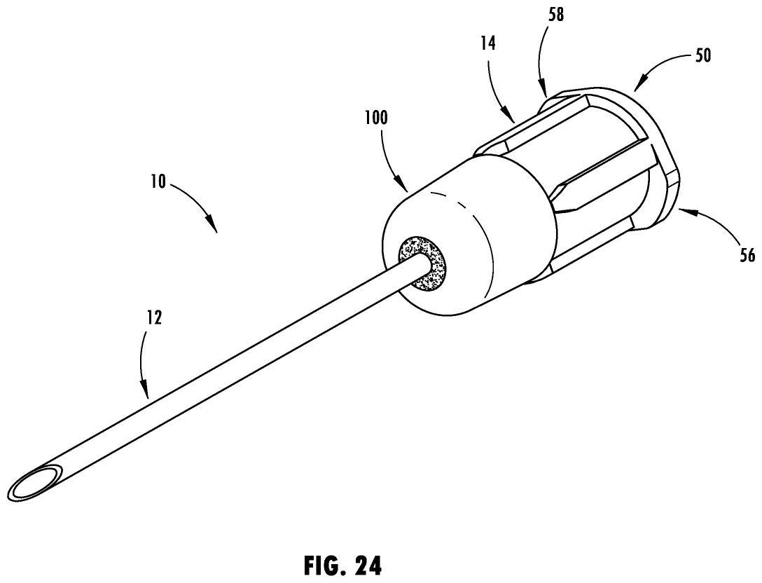

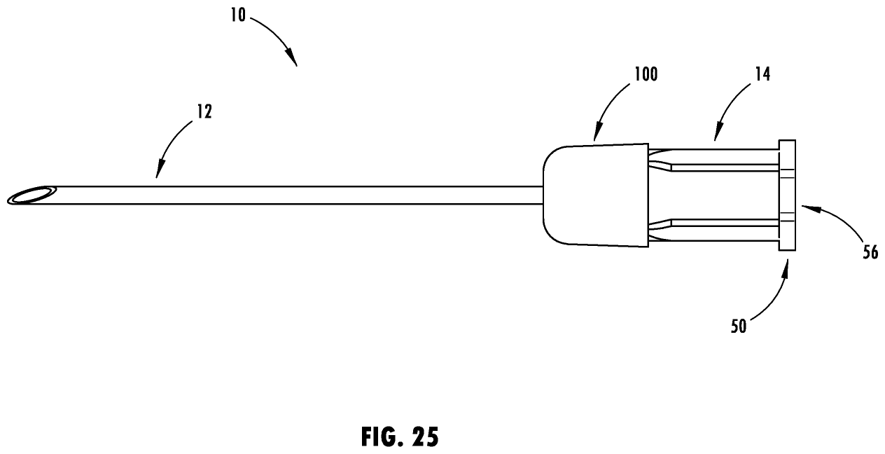

Referring to FIGS. 1, 19E, and 20-25, a hypodermic interface assembly 10 including a cannula 12 (see, e.g., FIGS. 1-2), a hub 14 (see, e.g., FIGS. 1 and 3-10), a cannula carrier 100 (see, e.g., FIGS. 1 and 11-17), and one or more fastening portions 200 (see, e.g., FIGS. 1 and 19A-19E) is shown. A central axis A₁₀-A₁₀ that extends through an axial center of each component (i.e., the cannula 12, the hub 14, the cannula carrier 100, and the one or more fastening portions 200) of the assembly 10 is shown extending through the hypodermic interface assembly 10.

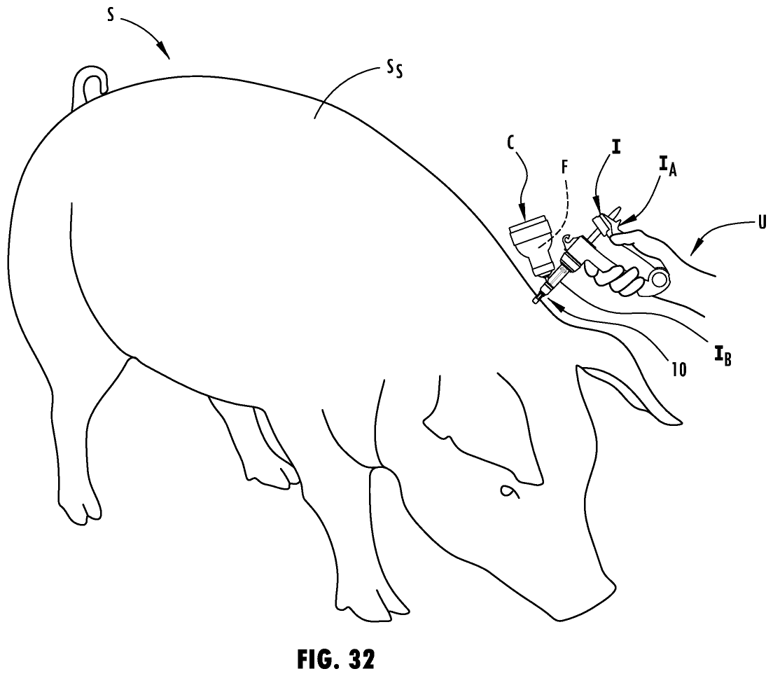

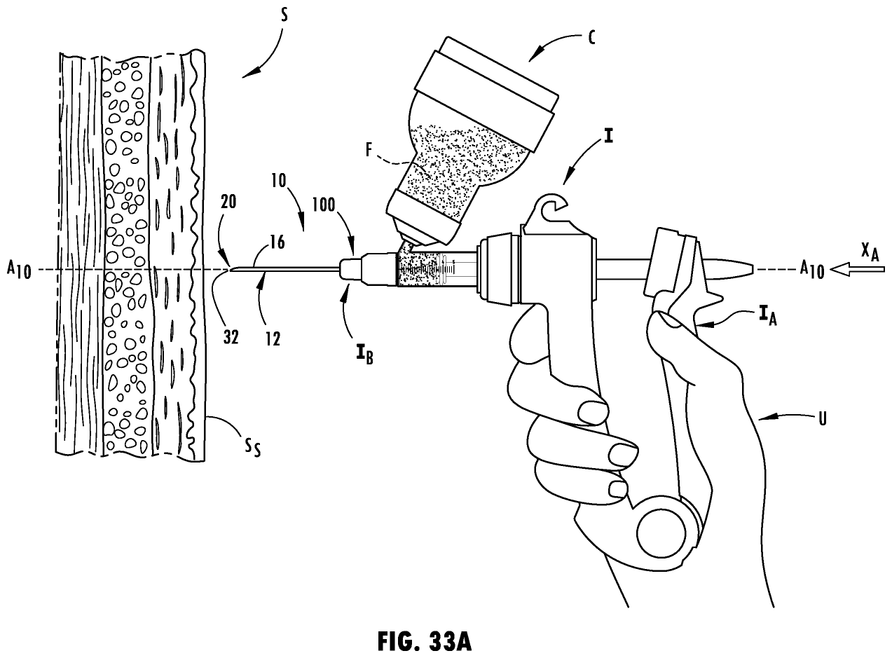

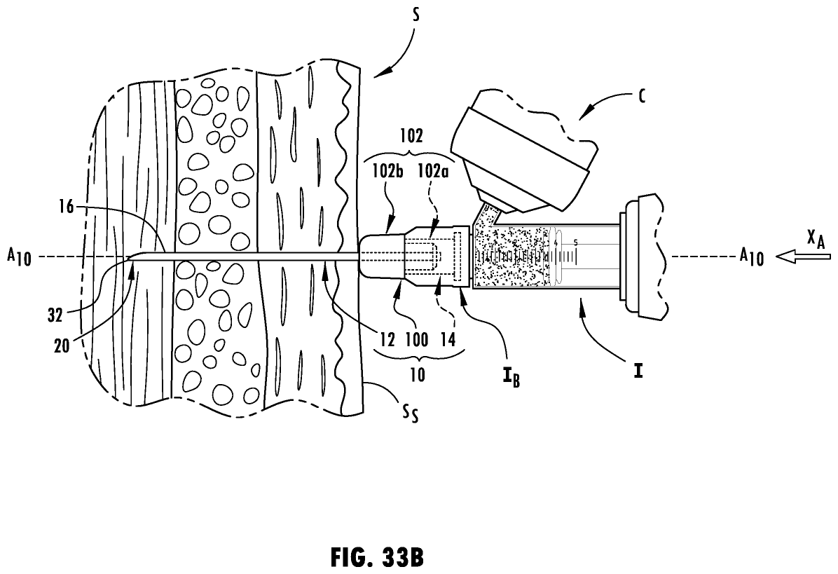

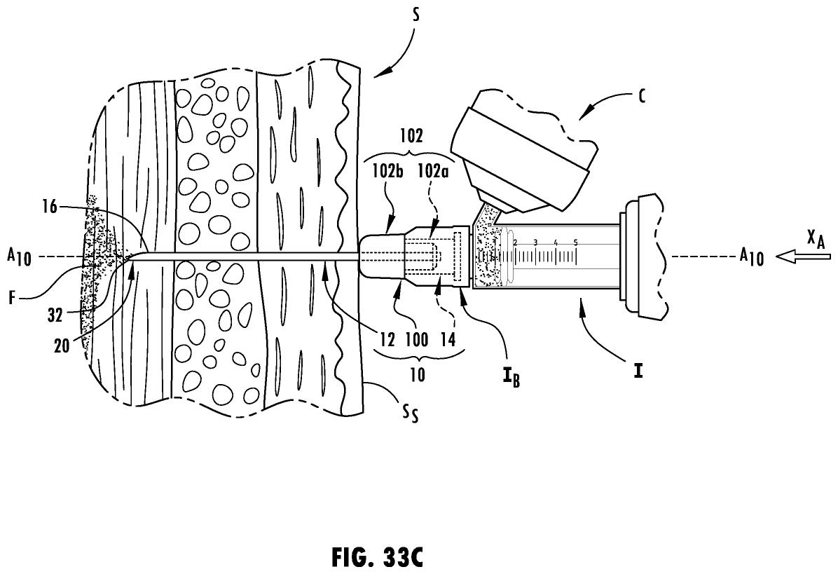

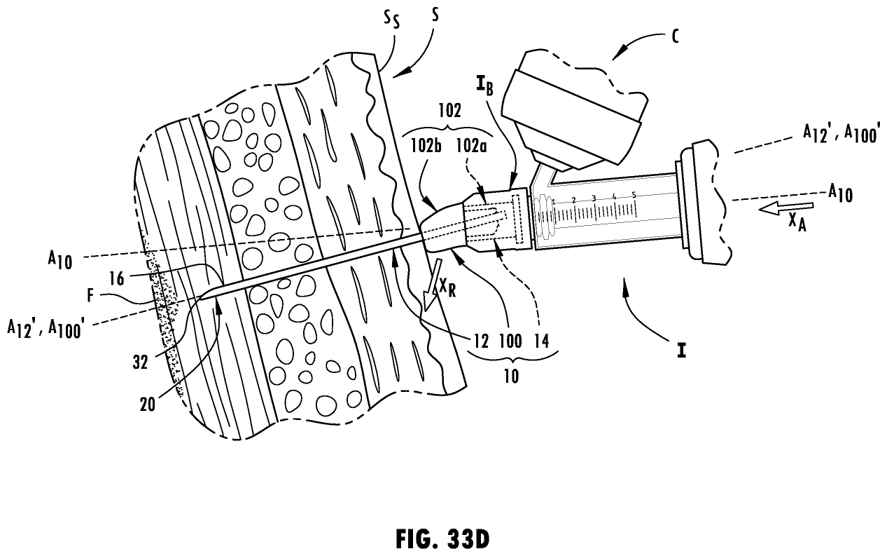

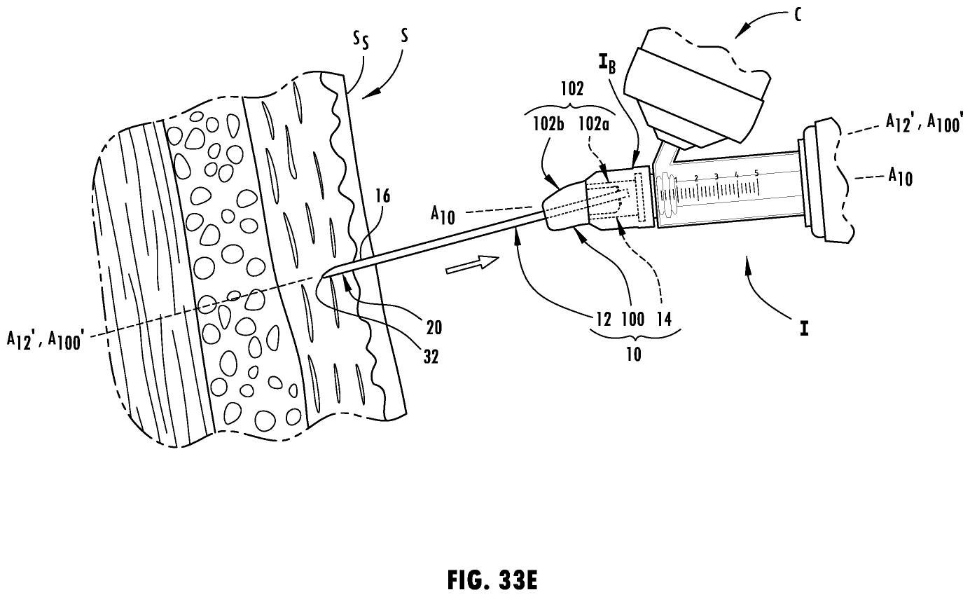

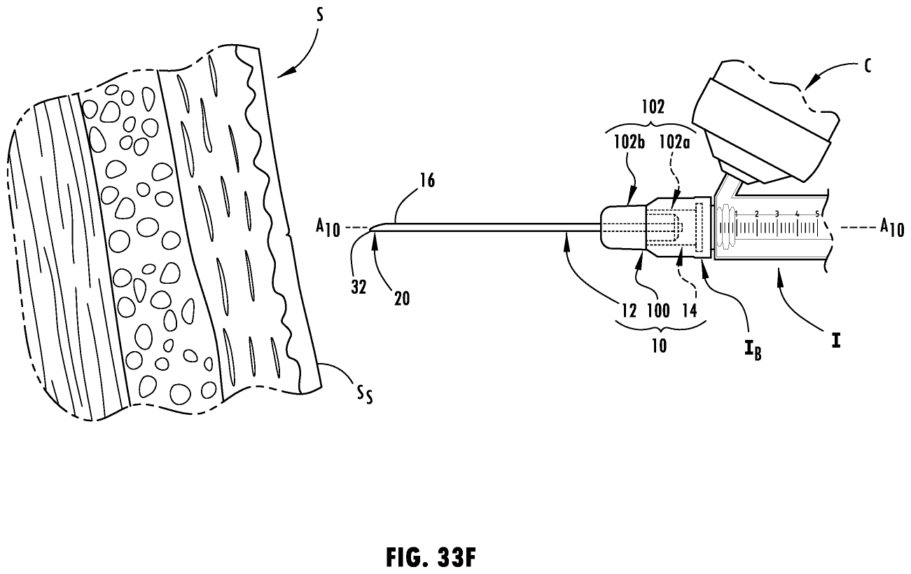

As seen at FIGS. 32 and 33A-33F, the cannula 12 is configured to pierce an outer surface S_{S} (e.g., the skin or hide) of a subject S (e.g., animalia, such as a human or non-human). The purpose of piercing the skin or hide S_{S} of the animalia S may be directed to injecting a fluid F (e.g., a medicament, a pharmaceutical, a vaccine, an anesthetic, or the like) into the animalia S. In other examples, the purpose of piercing the skin or hide S_{S} of the animalia S may be directed to the purpose of drawing a fluid F (e.g., blood) from the animalia S. Accordingly, the cannula 12 may be referred to as a hypodermic cannula, and, as such, the assembly 10 may be referred to as a hypodermic interface assembly as a result of the cannula 12 being capable of injecting or drawing a fluid F into/from the animalia S.

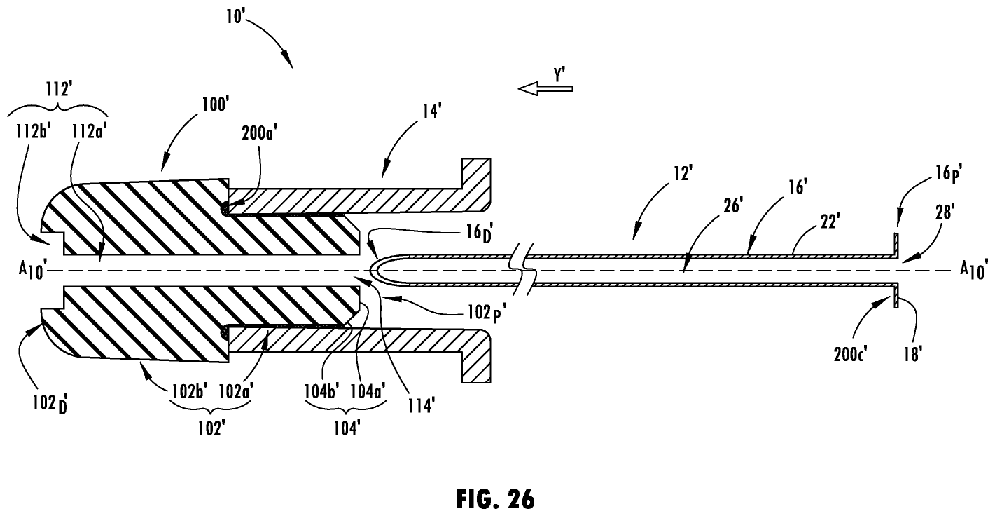

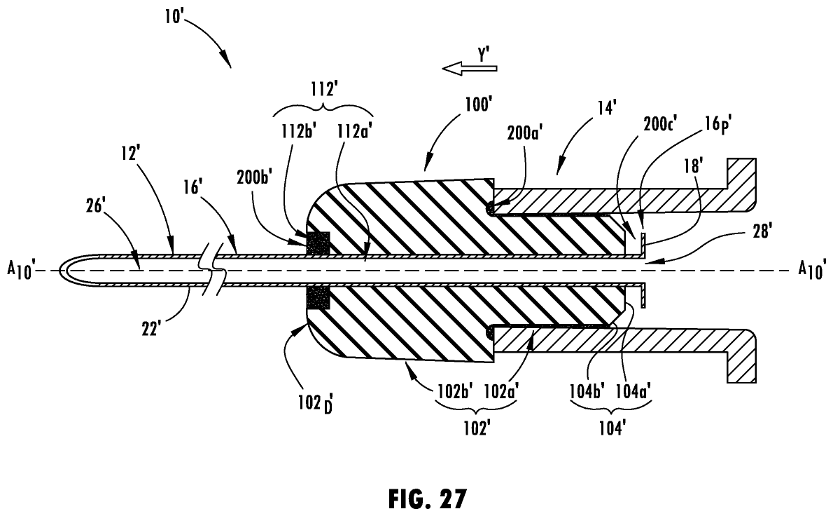

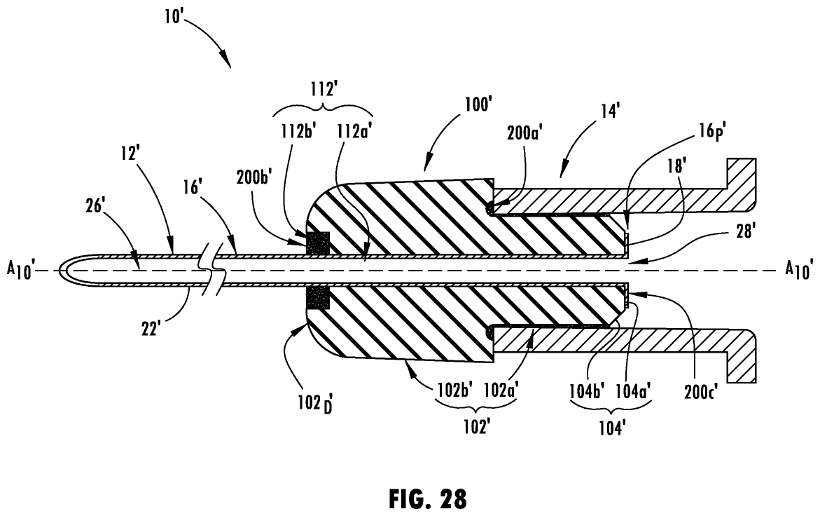

Although the one or more fastening portions 200 is/are shown and described below to include a first fastening portion 200a, a second fastening portion 200b, and a third fastening portion 200c, some configurations of the hypodermic interface assembly may be practiced using one or two (of the three illustrated and described) fastening portion 200a, 200b, 200c of the one or more fastening portions 200; accordingly, although each of the fastening portions 200a, 200b, 200c may be described below to be a ‘first,’ ‘second,’ or ‘third’ fastening portion, it should not be construed to mean or imply that a mentioning of, for example, a ‘second’ fastening portion requires a ‘first’ fastening portion in an exemplary design of the hypodermic interface assembly 10. As such, one or two of the fastening portions 200a, 200b, 200c may be omitted from the design of exemplary implementations of the hypodermic interface assembly 10 (i.e., exemplary implementations of the hypodermic interface assembly may include one, two, or all three of the fastening portions 200a, 200b, 200c). Furthermore, as will described in the following disclosure at FIGS. 26-28 and 29-31, some configurations of the hypodermic interface assembly 10 may not include any of the one or more fastening portions 200. As such, the cannula 12, the hub 14, and the cannula carrier 100 may be connected in a variety of manners or configurations.

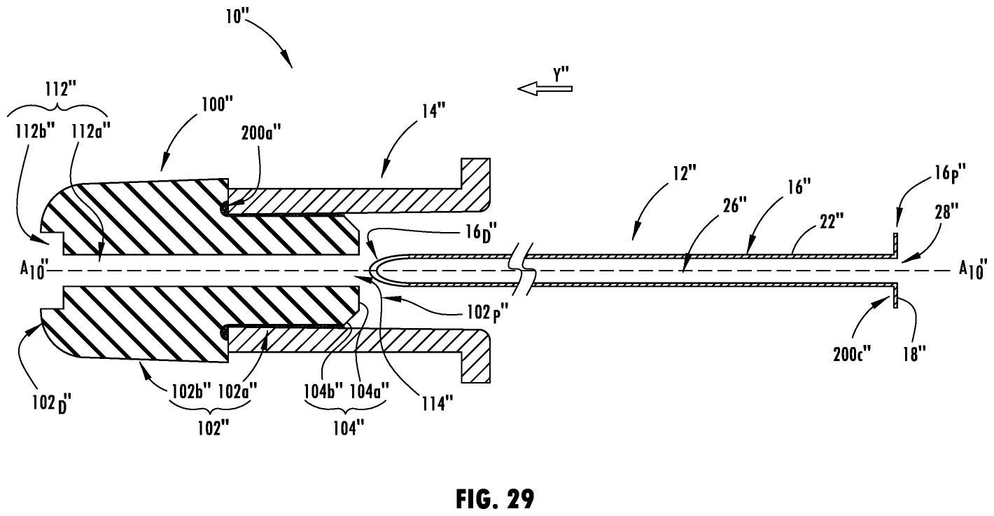

The design of any of the exemplary hypodermic interface assemblies 10 (and, see also, the hypodermic interface assemblies 10′, 10″ at FIGS. 26-28 and 29-31) promotes predictable and controlled bending or flexing of one or more portions of the cannula carrier 100 instead of the cannula 12 relative the hub 14 (see, e.g., FIG. 33D). In some instances, predictable and controlled bending or flexing of one or more portions of the cannula carrier 100 relative the hub 14 may occur after the cannula 12 pierces the subject S (see, e.g., FIGS. 29 and 33B-33D). The subject S may be, for example, animalia, such as a human or non-human (i.e., an animal, such as a pig or swine). In other examples, the subject S may be an inanimate object. The predicable and controlled bending or flexing of one or more portions of the cannula carrier 100 relative the hub 14 mitigates separation of the cannula 12 from the hub 14, which may otherwise result in the cannula 12 being broken-off and subsequently being lost within the flesh of the animalia.

As seen at FIG. 2, the cannula 12 is defined by a tube-shaped body 16 having a proximal end 16_{P} and a distal end 16_{D}. The cannula 12 is defined by a length L₁₂ extending between the proximal end 16_{P} of the tube-shaped body 16 and the distal end 16_{D} of the tube-shaped body 16. The length L₁₂ of the cannula 12 is defined by a plurality of sub-lengths L₁₂ₐ (including sub-length portions L₁₂ₐ₁ and L₁₂ₐ₂), L_{12b} (including sub-length portions L_{12b1}, L_{12b2}, and L_{12b3}), which will be further described in the following disclosure.

The cannula 12 may be formed using any desirable manufacturing procedure such as, for example: cold drawing of a larger tube, heated drawings, a molding procedure; a casting procedure; a machining procedure; a lathing procedure; or a combination thereof. The cannula 12 made from any desirable material such as, for example: a metallic material; a plastic material; or a combination thereof. In some examples, the cannula 12 may be made from a stainless steel material. In other instances, the cannula 12 may be made from an aluminum material or a ceramic material. In yet other examples, the cannula 12 may be made from a magnetically-detectable material or an ax-ray opaque material.

The cannula 12 may be defined in terms of ‘gauge size’ that takes into consideration skid/hide thickness of the subject S and/or a depth of injection of the subject S. The gauge size of the cannula 12 may be defined in a series of industry standard numbers in which, for example, the lower the number, the wider the diameter of the cannula. Furthermore, the series of industry standard numbers defining gauge size of the cannula 12 may be defined in a manner such that, for example, a higher gauge number indicates a smaller width of the cannula 12. In some instances, the industry standard gauge sizes of the cannula 12 may range from, for example: 14-Gauge; 16-Gauge; 18-Gauge; and 20-Gauge. Accordingly, in the range of exemplary industry standard numbers described above, a 14-Gauge cannula may be said to have a relatively largest diameter and highest strength (in terms of bendability/flexibility to a point where the cannula 12 could potentially break/fail) whereas a 20-Gauge cannula may be said to have a relatively smallest diameter and lowest strength (in terms of bendability/flexibility to a point where the cannula 12 could potentially break/fail).

A central axis A₁₂-A₁₂ extends through an axial center of the tube-shaped body 16 and along the length L₁₂ of the tube-shaped body 16. As will be described in the following disclosure at FIGS. 33D-33E and as seen at FIG. 2, a portion of the length Ln of the tube-shaped body 16 is permitted to bend, flex, or deviate from the central axis A₁₂-A₁₂ extends through an axial center of the tube-shaped body 16 and along the length Ln of the tube-shaped body 16. The portion of the length Lu of the tube-shaped body 16 that is permitted to bend, flex, or deviate from the central axis A₁₂-A₁₂ may be defined by, for example, the sub-length L₁₂ₐ₂ of the length L₁₂ of the tube-shaped body 16; accordingly, a portion of the central axis A₁₂-A₁₂ that extends through the bent or flexed sub-length L₁₂ₐ₂ of the length L₁₂ of the tube-shaped body 16 is seen generally at A₁₂′-A₁₂′. As seen at FIGS. 33D-33E, the axis A₁₂′-A₁₂′ may be said to be not aligned with and deviate away from the central axis A₁₀-A₁₀ extending through the hypodermic interface assembly 10 when the cannula 12 is arranged as a component of the hypodermic interface assembly 10.

The tube-shaped body 16 is further defined by a proximal end surface 18 at the proximal end 16_{P} of the tube-shaped body 16 and a distal end surface 20 at distal end 16_{D} of the tube-shaped body 16. The tube-shaped body 16 is further defined by an outer surface 22 extending between the proximal end surface 18 and the distal end surface 20. The tube-shaped body 16 is further defined by an inner surface 24 extending between the proximal end surface 18 and the distal end surface 20. The inner surface 24 further defines a passage 26 extending through the tube-shaped body 16. The proximal end surface 18 defines a proximal opening 28 that is in fluid communication with the passage 26. The distal end surface 20 defines a distal opening 30 that is in fluid communication with the passage 26.

With reference to FIG. 19E, which illustrates an enlarged cross-section view of an exemplary hypodermic interface assembly 10, the body 16 of the cannula 12 is defined by a thickness T₁₂ extending between the outer surface 22 of the body 16 and the inner surface 24 of the body 16. The outer surface 22 further defines an outer diameter D₁₂ of the cannula 12 that is referenced from the central axis A₁₂-A₁₂, which may be coincident with respective central axes A₁₀-A₁₀, A₁₄-A₁₄, and A₁₀₀-A₁₀₀ of each of the hypodermic interface assembly 10, the hub 14, and the cannula carrier 100. The inner surface 24 further defines the passage 26 to have a passage diameter D₂₆. The passage 26 is in fluid communication with the proximal opening 28 and the distal opening 30 in order to permit: (1) passage of a fluid F (see, e.g., FIG. 33C) into the tube-shaped body 16 at the proximal opening 28; (2) through the passage 26 in a direction from the proximal end 16_{P} of the tube-shaped body 16 and towards the distal end 16_{D} of the tube-shaped body 16; and (3) out of the distal opening 30.

With reference to FIGS. 2 and 19E, the proximal end surface 18 extends from the outer surface 22 substantially perpendicularly, and, as such, defines the proximal end surface 18 to be blunted or non-sharpened. Furthermore, the proximal opening 28 formed by the proximal end surface 18 may define a substantially circular-shaped geometry that is defined by a proximal opening diameter D₂₈ that is substantially similar to the passage diameter D₂₆ of the passage 26.

With reference to FIG. 2, the distal end surface 20 extends from the outer surface 22 at a beveled angle θ₂₀, and, as such, the distal end surface 20 may be referred to as a beveled distal end surface that terminates at or defines a sharp piercing tip 32. The beveled distal end surface 20 may be defined by any desirable beveled angle θ₂₀ that forms, for example, a “standard bevel,” a “short bevel,” or a “true short bevel.” Because the beveled distal end surface 20 extends from the outer surface 22 at a beveled angle θ₂₀, the distal opening 30 may be defined by an oval-shaped geometry. The distal end 16_{D} of the tube-shaped body 16 of the cannula 12 may be defined by any desirable bevel orientation; for example, three bevels may be utilized in order to maintain sharpness of the cannula 12 and/or to avoid an obstruction, plugging, or clogging of passage 26 extending through the cannula 12.

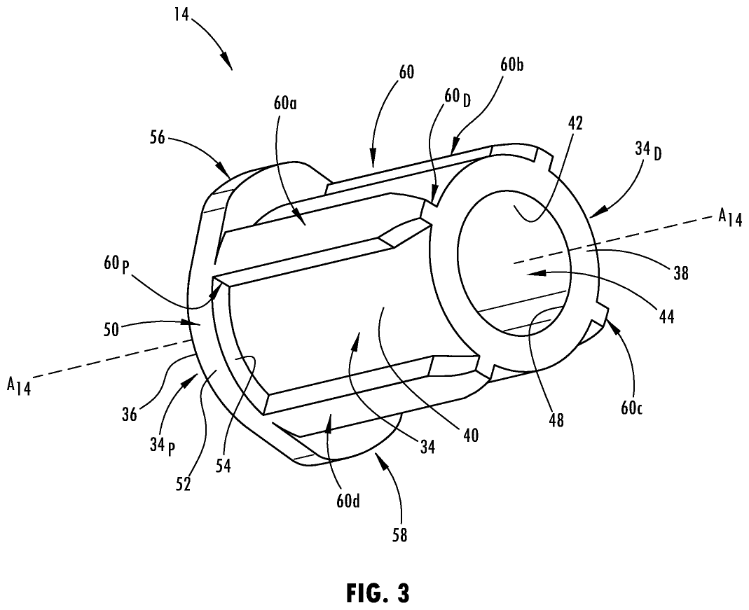

As seen at FIGS. 3-10, the hub 14 is defined by a substantially tube-shaped body 34 having a proximal end 34_{P} and a distal end 34_{D}. The hub 14 is defined by a length L₁₄ (see, e.g., FIG. 7) extending between the proximal end 34_{P} of the substantially tube-shaped body 34 and the distal end 34_{D} of the substantially tube-shaped body 34. The length L₁₄ of the hub 14 is defined by a plurality of sub-lengths L₁₄ₐ (including sub-length portions L₁₄ₐ₁ and L₁₄ₐ₂), L_{14b}, and L_{14c}, which will be further described in the following disclosure.

The hub 14 may be formed using any desirable manufacturing procedure such as, for example: a molding procedure; a casting procedure; a machining procedure; a lathing procedure; or a combination thereof. The hub 14 made from any desirable material such as, for example: a metallic material; a plastic material; or a combination thereof. In some examples, the hub 14 may be made from a stainless steel material. In other instances, the hub 14 may be made from an aluminum material or a brass material. The hub 14 may alternatively be made from a polymeric material, such as, for example, polypropylene, polyurethane, polyester, polystyrene, or the like.

The substantially tube-shaped body 34 is further defined by a proximal end surface 36 at the proximal end 34_{P} of the substantially tube-shaped body 34 and a distal end surface 38 at distal end 34_{D} of the substantially tube-shaped body 34. The substantially tube-shaped body 34 is further defined by an outer surface 40 extending between the proximal end surface 36 and the distal end surface 38. The substantially tube-shaped body 34 is further defined by an inner surface 42 extending between the proximal end surface 36 and the distal end surface 38.

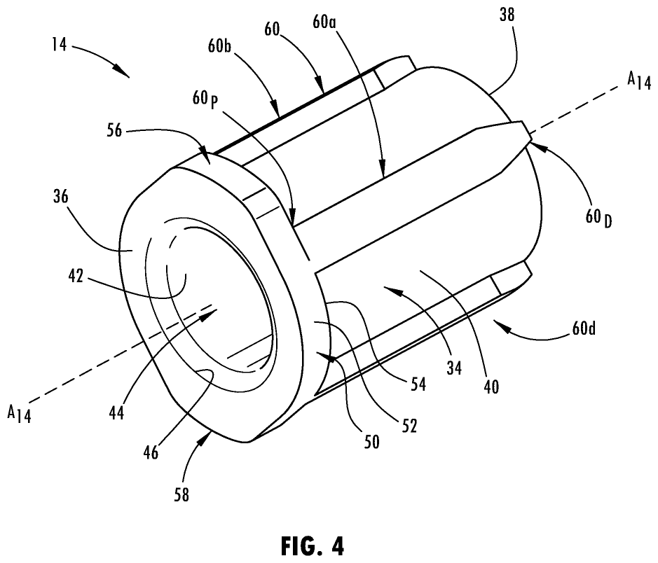

The inner surface 42 further defines a passage 44 extending through the substantially tube-shaped body 34. The proximal end surface 36 defines a proximal opening 46 (see, e.g., FIGS. 4, 7, and 10) that is in fluid communication with the passage 44. The distal end surface 38 defines a distal opening 48 (see, e.g., FIGS. 3, 5, 7, 8B, 9B) that is in fluid communication with the passage 44.

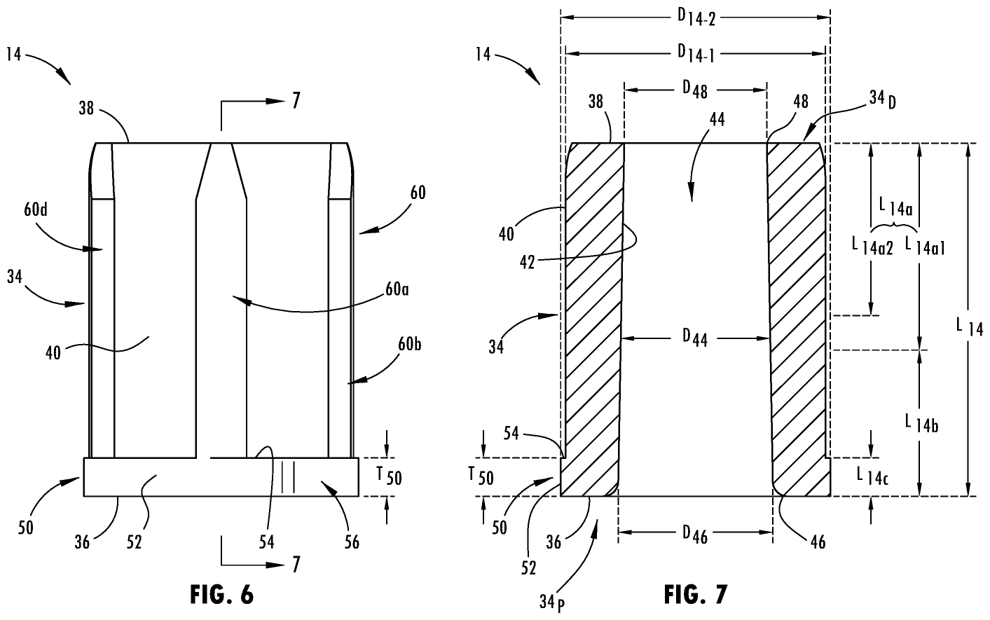

As seen at FIGS. 3-10, a ring portion 50 projects radially outwardly away from a central axis A₁₄-A₁₄ away from the outer surface 40 of the substantially tube-shaped body 34. The ring portion 50 may be alternatively referred to as a barrel-engaging portion that is configured to be connected to a barrel portion I_{B} of an injection gun I (see, e.g., FIG. 32). The barrel-engaging portion 50 is defined by an outer side surface 52 that extends between the proximal end surface 36 and a distal shoulder surface 54. The barrel-engaging portion 50 may be defined by a thickness T₅₀ (see, e.g., FIGS. 6 and 7) extending between the proximal end surface 36 and the distal shoulder surface 54.

The outer surface 40 of the substantially tube-shaped body 34 may define a substantially circular-shaped geometry that defines a first outer diameter D₁₄₋₁ (see, e.g., FIG. 7) of the hub 14. The outer side surface 52 of the barrel-engaging portion 50 may define a substantially circular-shaped geometry that defines a second outer diameter D₁₄₋₂ (see, e.g., FIG. 7) of the hub 14. The second outer diameter D₁₄₋₂ of the hub 14 is greater than the first outer diameter D₁₄₋₁ of the hub 14.

As seen at FIGS. 3-5, 8A-8B, 9A-9B, and 10, the substantially circular-shaped geometry of the outer side surface 52 of the barrel-engaging portion 50 is interrupted by a first radially-outward projection or ear 56 and a second radially-outward projection or ear 58 that extend beyond the second outer diameter D₁₄₋₂ of the hub 14. The first radially-outward projection or ear 56 may be arranged opposite of or offset approximately 180° from the second radially-outward projection or ear 58.

As seen at FIG. 7, the inner surface 42 of the substantially tube-shaped body 34 defines a passage diameter D₄₄ (see, e.g., FIG. 7) of the passage 44. The passage diameter D₄₄ is greater than the outer diameter D₁₂ of the cannula 12.

The passage 44 is in fluid communication with the proximal opening 46 and the distal opening 48 in order to permit: (1) passage of a fluid F (see, e.g., FIG. 33C) into the substantially tube-shaped body 34 at the proximal opening 46; (2) through the passage 44 in a direction from the proximal end 34_{P} of the substantially tube-shaped body 34 and towards the distal end 34_{D} of the substantially tube-shaped body 34; and (3) out of the distal opening 48.

The proximal opening 46 formed by the proximal end surface 36 may define a substantially circular-shaped geometry that is defined by a proximal opening diameter D₄₆ (see, e.g., FIG. 7) that is substantially similar to the passage diameter D₄₄ of the passage 44. The distal opening 48 formed by the distal end surface 38 may define a substantially circular-shaped geometry that is defined by a distal opening diameter D₄₈ (see, e.g., FIG. 7) that is substantially similar to the diameter D₄₄ of the passage 44.

Referring to FIGS. 3-6, 8A, 8B, 9A, and 9B, one or more ribs 60 may project radially outwardly away from a central axis A₁₄-A₁₄ away from the outer surface 40 of the substantially tube-shaped body 34. The one or more ribs 60 may include, for example, a first rib 60a, a second rib 60b, a third rib 60c, and a fourth rib 60d.

The one or more ribs 60 may increase the structural integrity of the substantially tube-shaped body 34 of the hub 14. In some configurations, the one or more ribs 60 may arise from mold relief features during the manufacturing process of the substantially tube-shaped body 34 of the hub 14.

Each rib 60a, 60b, 60c, 60d of the one or more ribs 60 includes a distal end 60_{D} and a proximal end 60_{P}. The proximal end 60_{P} of each rib 60a, 60b, 60c, 60d of the one or more ribs 60 extends from the distal shoulder surface 54 of the barrel-engaging portion 50. The distal end 60_{D} of each rib 60a, 60b, 60c, 60d of the one or more ribs 60 extends from the distal end surface 38 of the substantially tube-shaped body 34. Each rib 60a, 60b, 60c, 60d of the one or more ribs 60 may define a substantially rectangular body that terminates with a substantially triangular body portion defined by the distal end 60_{D} of each rib 60a, 60b, 60c, 60d of the one or more ribs 60.

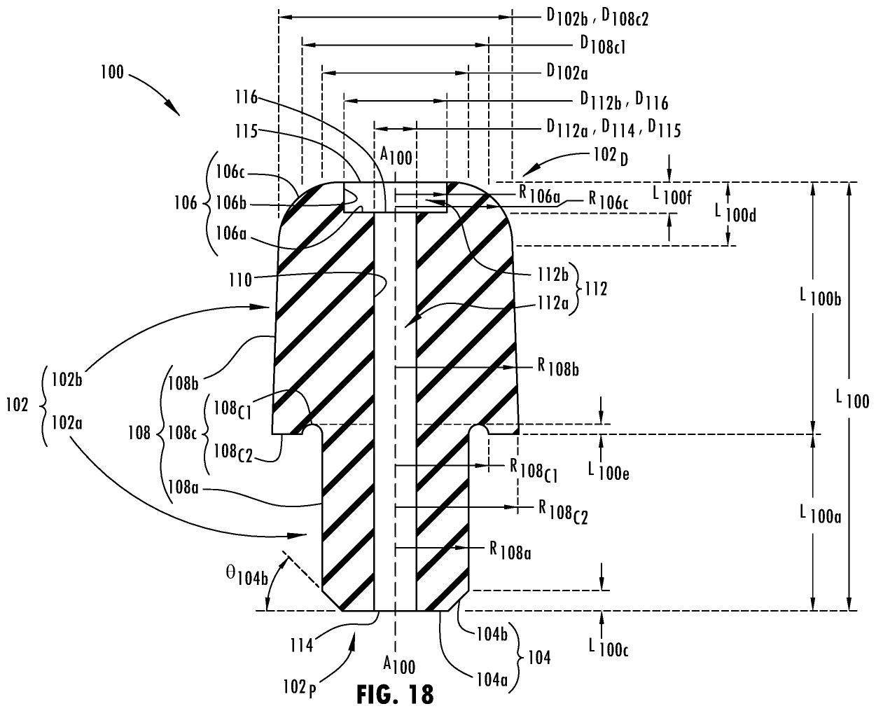

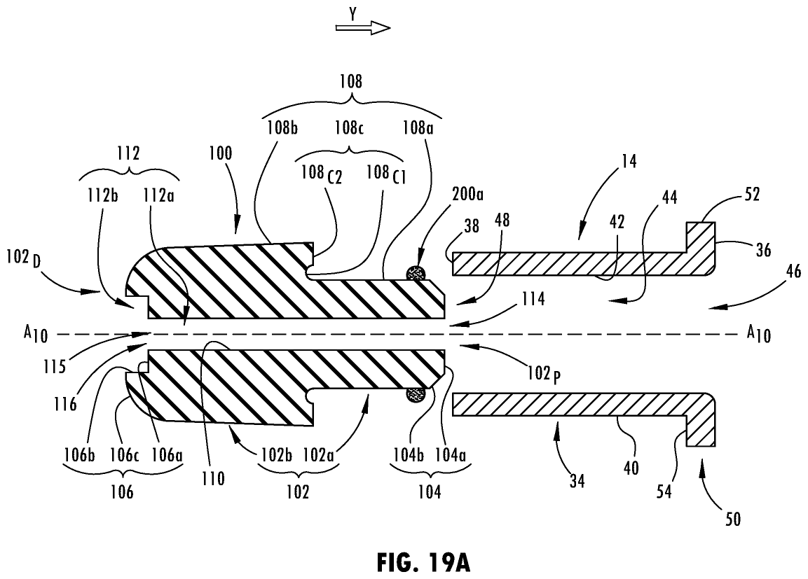

As seen at FIGS. 11-18, the cannula carrier 100 is defined by a substantially tube-shaped body 102 having a proximal end 102_{P} and a distal end 102_{D}. The cannula carrier 100 is defined by a length L₁₀₀ (see, e.g., FIG. 18) extending between the proximal end 102_{P} of the substantially tube-shaped body 102 and the distal end 102_{D} of the substantially tube-shaped body 102. The length L₁₀₀ of the cannula carrier 100 is defined by a plurality of sub-lengths L₁₀₀ₐ, L_{100b}, L_{100c}, L_{100d}, L₁₀₀ₑ, L_{100f}, which will be further described in the following disclosure.

The cannula carrier 100 may be formed from a flexible material that permits one or both of a neck portion 102a and a head portion 102b of the substantially tube shaped body 102 of the cannula carrier 100 to bend, flex, or deviate away from a central axis A₁₀₀-A₁₀₀ of the cannula carrier 100. With reference to FIG. 17, as an example and described in the following disclosure at FIGS. 33D-33E, a portion of the length L₁₀₀ of the substantially tube-shaped body 102 is permitted to bend, flex, or deviate from a central axis A₁₀₀-A₁₀₀ that extends through an axial center of the tube-shaped body 102 and along the length L₁₀₀ of the tube-shaped body 102. The portion of the length L₁₀₀ of the tube-shaped body 102 that is permitted to bend, flex, or deviate from the central axis A₁₀₀-A₁₀₀ may be defined by, for example, the sub-length L₁₀₀b of the length L₁₀₀ of the tube-shaped body 102; accordingly, a portion of the central axis A₁₀₀-A₁₀₀ that extends through the bent or flexed sub-length L₁₀₀b of the length L₁₀₀ of the tube-shaped body 102 is seen generally at A₁₀₀′-A₁₀₀′ at FIGS. 17, 19E′, 19E″ and 33D-33E. As seen at FIGS. 19E′, 19E″ and 33D-33E, the axis A₁₀₀′-A₁₀₀′ may be said to be not aligned with and deviate away from the central axis A₁₀-A₁₀ extending through the hypodermic interface assembly 10 when the cannula carrier 100 is arranged as a component of the hypodermic interface assembly 10.

With reference to FIGS. 19E′, 19E″ and 33D, in some instances, when, for example, the cannula 12 being subjected to one or more radial forces X_{R} relative to the central axis A₁₀-A₁₀ extending through the hypodermic interface assembly 10 when the cannula 12 is connected to the cannula carrier 100, the head portion 102b of the substantially tube-shaped body 102 of the cannula carrier 100 radially flexes or bends away from the central axis A₁₀₀-A₁₀₀ of the cannula carrier 100 (and defined as the axis A₁₀₀′-A₁₀₀′ of the cannula carrier 100) relative to the neck portion 102a of the substantially tube-shaped body 102 of the cannula carrier 100, which may be secured to the hub 14; while the neck portion 102a of the substantially tube-shaped body 102 of the cannula carrier 100 may not flex or bend away from the central axis A₁₀₀-A₁₀₀ of the cannula carrier 100. Accordingly, the head portion 102b of the substantially tube-shaped body 102 of the cannula carrier 100 may define a deformable portion of the substantially tube-shaped body 102 of the cannula carrier 100 that relieves radial stresses imparted to the cannula 12 arising from movement of a subject S during a probing or injecting procedure (see e.g., FIGS. 33B-33D) associated with the sharp piercing tip 32 formed by the distal end surface 20 of the tube-shaped body 16 of the cannula 12 piercing an outer surface S_{S} (e.g., the skin or hide) of the subject S. Accordingly, a location of stress concentration applied to the cannula 12 arising from one or more radial forces X_{R} that is/are imparted to the cannula 12 may be axially moved away from the hub 14 and closer to the outer surface S_{S} of the subject S.

The cannula carrier 100 may be formed using any desirable manufacturing procedure such as, for example, a molding procedure, a casting procedure, room temperature curing, or 3D printing. The cannula carrier 100 made from any desirable material such as, for example, a plastic material. In some examples, the cannula carrier 100 may be made from a rubber material. In other instances, the cannula carrier 100 may be made from a polymeric material. In some implementations, the cannula carrier 100 may be made from a poly-vinyl-chloride (PVC) material. In other configurations, the cannula carrier 100 may be made from a polypropylene co-polymer. The cannula carrier 100 may alternatively be made from a vulcanized natural latex rubber or any desirable polymeric or a copolymeric material such as, for example, polyisoprene, polystyrene cobutadiene, polyethylene covinylacetate, or the like. In other implementations, the cannula carrier 100 may be made from a thermosetting material such as, for example, polyurethane, polyesters, or silicone rubber.

Irrespective of the type of material selected for forming the cannula carrier 100, the material may be defined by a hardness that may be quantified in terms of a Shore durometer hardness. There are three Shore durometer ranges: Shore 00 scale may be used to describe very soft materials such as marshmallows (Shore 00 of 10) Insole gels (Shore 00 of 30). Shore A scale is for intermediate materials. A typical mousepad may be about Shore A 20 durometer (“20A”), and an infant feeding bottle nipple might be Shore A 40 durometer. A smartwatch band be Shore A 70 durometer, and a typical leather belt might be around Shore A 80 durometer. The rubber on a shopping cart wheel might be Shore A >90 durometer. Shore D durometer range is for still harder materials. The rubber on a shopping cart wheel might for instance have a Shore D of 50, The popular construction toy Lego® might have a Shore D durometer of 60, while a PVC schedule 40 plumbing joint might have a durometer Shore D of 80. In one embodiment, the type of material selected for forming the cannula carrier 100 may be defined by a Shore A durometer ranging between approximately (˜) 80-95. In some instances, the Shore A durometer of the cannula carrier 100 may be ˜80, ˜85, ˜90, or ˜95. The durometer (propensity to deform by a given stress, also known as “hardness” “flexibility” “modulus”) may be controlled by altering various polymeric characteristics. These include degree of cross-linking, degree of association of various domains, degree of order within the polymer, polymeric tacticity, polymer molecular weight, polymer molecular weight distribution, ratios of copolymers, and block vs. random copolymers.

KRATON®, for example, is a thermoplastic elastomer containing rigid and flexible sections of a block-copolymer made by Kraton Corporation. SANTOPRENE® is yet another exemplary thermoplastic elastomer that is commercially available from ExxonMobil. The durometer can further be impacted by materials ad-mixed into the polymer, including blending other polymers and or copolymers into the elastomeric polymer, adding a filler such as clays, calcium carbonate, carbon black, titanium dioxide, silica, modified silica, talc, and other particulates. Exemplary polymer resins can be made with specific durometer ranges as seen below in TABLE 1.

TABLE 1

| Shore A Durometer | Polymer resin commercial name |
|---|---|
| 79A-81A | MEDALIST ® MD-53278 |
| 84A-86A | MEDALIST ® MD-53283 |
| 89A-90A | MEDALIST ® MD-53288 |
| 93A-94A | MEDALIST ® MD-53293 |

The selected Shore durometer of the cannula carrier 100 may also correspond to the gauge size of the cannula 12. In some examples, a length and/or gauge size of the cannula 12 that is 14-Gauge may correspond to a selected Shore 85A-95A durometer of the cannula carrier 100. In other examples, a length and/or gauge size of the cannula 12 that is 16-Gauge may correspond to a selected Shore 80A-95A durometer of the cannula carrier 100. In further examples, a length and/or gauge size of the cannula 12 that is 20-Gauge may correspond to a selected Shore 75A-85A durometer of the cannula carrier 100.

The substantially tube-shaped body 102 is further defined by a proximal end surface 104 at the proximal end 102_{P} of the substantially tube-shaped body 102 and a distal end surface 106 at distal end 102_{D} of the substantially tube-shaped body 102. The substantially tube-shaped body 102 is further defined by an outer surface 108 extending between the proximal end surface 104 and the distal end surface 106. The substantially tube-shaped body 102 is further defined by an inner surface 110 extending between the proximal end surface 104 and the distal end surface 106.

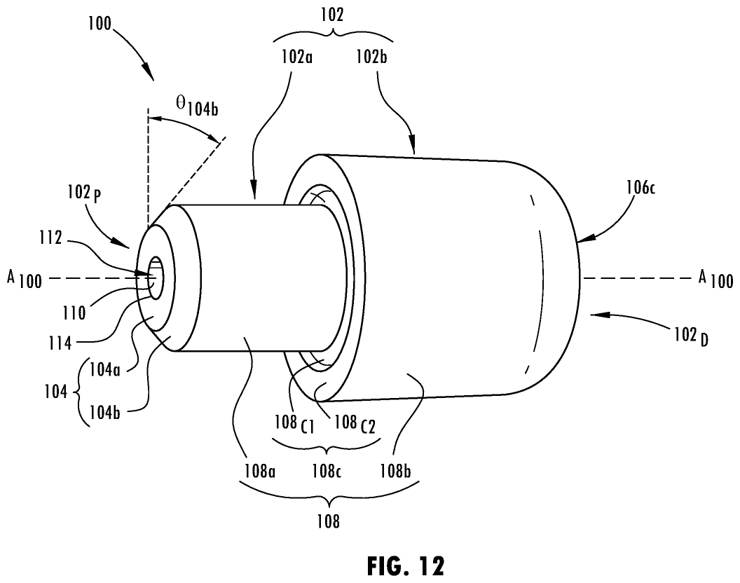

As seen at FIG. 18, the inner surface 110 further defines a passage 112 extending through the substantially tube-shaped body 102. The proximal end surface 104 defines a proximal opening 114 (see also, e.g., FIGS. 12, 13, and 16) that is in fluid communication with the passage 112. The distal end surface 106 defines a distal opening 116 (see also, e.g., FIGS. 11, 14, 15, and 16) that is in fluid communication with the passage 112.

Referring to FIGS. 12, 13, 16, 17 and 18, the proximal end surface 104 is defined by a first proximal end surface portion 104a and a second proximal end surface portion 104b. The first proximal end surface portion 104a extends substantially perpendicularly from the inner surface 110 of the cannula carrier 100. The second proximal end surface portion 104b extends at an angle θ_{104b} (see, e.g., FIGS. 12, 13, 17, and 18) from the first proximal end surface portion 104a. In some instances, the angle θ_{104b} may be approximately equal to 45°; accordingly, the second proximal end surface portion 104b may define a conical portion of the proximal end surface 104 that terminates with a flat portion defined by the first proximal end surface portion 104a.

Referring to FIGS. 11, 14, 15, and 18, the distal end surface 106 is defined by a first distal end surface portion 106a, a second distal end surface portion 106b, and a third distal end surface portion 106c. The first distal end surface portion 106a extends substantially perpendicularly from the inner surface 110 of the cannula carrier 100 as referenced from a radial distance R₁₀₆ₐ (see, e.g., FIG. 18) from the central axis A₁₀₀-A₁₀₀ extending through the axis center of the substantially tube-shaped body 102 of the cannula carrier 100. The second distal end surface portion 106b extends substantially perpendicularly from the first distal end surface portion 106a at a sub-length (see, e.g., sub-length L_{100f} of the length L₁₀₀ at FIG. 18). The third distal end surface portion 106c arcuately extends from the second distal end surface portion 106c as defined by an internal radius R_{106c} (see, e.g., FIG. 18) from the central axis A₁₀₀-A₁₀₀ extending through the axis center of the substantially tube-shaped body 102 of the cannula carrier 100; accordingly, the third distal end surface portion 106c may define a dome-shaped portion of the distal end surface 106 that terminates at a centrally recessed circular well (see, e.g., a region generally defined by reference numeral 112b). The recessed well may be defined in part by the first distal end surface portion 106a and the second distal end surface portion 106b that extends substantially perpendicularly from the first distal end surface portion 106a.

As will be described in the following disclosure and as seen at FIG. 18, the inner surface 110 may define a first passage portion 112a of the passage 112. Furthermore, the centrally recessed circular well (defined by the first distal end surface portion 106a and the second distal end surface portion 106b) may define a second passage portion 112b of the passage 112 that is in fluid communication with the first passage portion 112a of the passage 112.

Referring to FIGS. 11-14 and 16-18, the outer side surface 108 is defined by a first outer side surface portion 108a, a second outer side surface portion 108b, and a third outer side surface portion 108c. The first outer side surface portion 108a extends from the second proximal end surface portion 104b of the proximal end surface 104. The second outer surface portion 108b extends from the third distal end surface portion 106c of the distal end surface 106.

The third outer side surface portion 108c is arranged between and connects the first outer side surface portion 108a to the second outer side surface portion 108b. The third outer side surface portion 108c may extend substantially perpendicularly from each of the first outer side surface portion 108a and the second outer side surface portion 108b. Furthermore, the third outer side surface portion 108c is a proximal-side-facing shoulder surface that directly joins the first outer side surface portion 108a to the second outer side surface portion 108b. In other words, as described in greater detail below, the third outer side surface portion 108c functions as a shoulder surface that is configured to be disposed proximate (i.e., at least near), opposite (i.e., facing), or adjacent (i.e., in contact with) the distal end surface 38 of the substantially tube-shaped body 34 when hypodermic interface assembly 10 is assembled.

As discussed above and in the following disclosure, it will be appreciated that the limitation, “proximate,” could mean to be in contact with or spaced apart (e.g., by an air gap or an intervening layer of material, such as, for example, adhesive). Furthermore, the limitation, “opposite,” could mean to be in contact with or spaced apart (e.g., by an air gap or an intervening layer of material, such as, for example, adhesive). Yet even further, the limitation, “adjacent” could mean to be directly in contact with (such that opposing surfaces are touching one another), or indirectly in contact with (such that opposing surfaces are indirectly touching one another with, for example, an intervening layer of material, such as, for example, adhesive).

As seen at FIG. 18, the first outer side surface portion 108a extends away from a central axis A₁₀₀-A₁₀₀ of the cannula carrier 100 at a first radial distance R₁₀₈ₐ thereby defining a first outer diameter D₁₀₂ₐ of the substantially tube-shaped body 102. The second outer side surface portion 108b extends away from the central axis A₁₀₀-A₁₀₀ of the cannula carrier 100 at a second radial distance R_{108b} thereby defining a second outer diameter D_{102b} of the substantially tube-shaped body 102. The second radial distance R_{108b} is greater than the first radial distance R₁₀₈ₐ; accordingly, the first outer side surface portion 108a may be said to define a neck portion 102a of the substantially tube-shaped body 102, and the second outer side surface portion 108b may be said to define a head portion 102b of the substantially tube-shaped body 102.

As seen at FIGS. 17 and 18, the third outer side surface portion 108c may be referred to as a shoulder surface portion of the outer side surface 108 that joins the first outer side surface portion 108a to the second outer side surface portion 108b. Furthermore, with reference to FIG. 18, the third outer side surface portion 108c may be configured to define a first shoulder surface portion 108c₁ (see also FIGS. 12, 13, and 16) and a second shoulder surface portion 108c₂ (see also FIGS. 12, 13, and 16).

As seen at FIG. 18, the first shoulder surface portion 108c₁ extends from the second proximal end surface portion 104b of the proximal end surface 104 at a first radial distance R_{108c1} defining the first shoulder surface portion 108c₁ to define a first diameter D_{108c1} of the third outer side surface portion 108c. In some configurations, as seen at, for example, FIGS. 12, 13, and 16, the first shoulder surface portion 108c₁ defines an arcuate recess that circumscribes the neck portion 102a of the substantially tube-shaped body 102.

As seen at FIG. 18, the second shoulder surface portion 108c₂ extends from the third distal end surface portion 106c of the distal end surface 106 at a second radial distance R_{108c2} defining the second shoulder surface portion 108c₂ to define a second diameter D_{108c2} of the third outer side surface portion 108c. In some configurations, as seen at, for example, FIGS. 12, 13, and 16, the second shoulder surface portion 108c₂ is defined by the substantially flat surface of the third outer side surface portion 108c that surrounds the first shoulder surface portion 108c₁ that defines the arcuate recess of the third outer side surface portion 108c.

With continued reference to FIG. 18, the passage 112 is defined by a first passage diameter D₁₁₂ₐ and a second passage diameter D_{112b}. The second passage diameter D_{112b} is greater than the first passage diameter D₁₁₂ₐ. The first passage diameter D₁₁₂ₐ is defined by the inner surface 110 of the substantially tube-shaped body 102 that defines the first passage portion 112a of the passage 112. The second passage diameter D_{112b} is defined by the second distal end surface portion 106b of the substantially tube-shaped body 102 that partially defines the second passage portion 112b of the passage 112.

The first passage portion 112a of the passage 112 is in fluid communication with the proximal opening 114. The second passage portion 112b of the passage 112 is in fluid communication with the distal opening 116. Accordingly, the passage 112 permits: (1) flowing of a fluid F (see, e.g., FIG. 33C) into the substantially tube-shaped body 102 at the proximal opening 114; (2) through the passage 112 in a direction from the proximal end 102_{P} of the substantially tube-shaped body 102 and towards the distal end 102_{D} of the substantially tube-shaped body 102; and (3) out of the distal opening 116.

As seen at FIGS. 12, 13, 16, and 18, the proximal opening 114 formed by the proximal end surface 104 may define a substantially circular-shaped geometry that is defined by a proximal opening diameter D₁₁₄ (see, e.g., FIG. 18) that is substantially similar to the first passage diameter D₁₁₂ₐ of the first passage portion 112a of the passage 112. As seen at FIGS. 11, 14, 15, and 18, the distal opening 116 formed by the distal end surface 106 may define a substantially circular-shaped geometry that is defined by a distal opening diameter D₁₁₆ (see, e.g., FIG. 18) that is substantially similar to the second passage diameter D₁₁₂b of the second passage portion 112b of the passage 112.

As seen at FIGS. 11, 14, 15, and 18, an intermediate opening 115 is formed by the connection of the inner surface 110 to the first distal end surface portion 106a. The intermediate opening 115 may define a substantially circular-shaped geometry that is defined by an intermediate opening diameter D₁₁₅ (see, e.g., FIG. 18) that is substantially similar to the first passage diameter D₁₁₂ₐ of the first passage portion 112a of the passage 112.

As described above and as seen at FIG. 18, the length L₁₀₀ of the cannula carrier 100 is defined by a plurality of sub-lengths L₁₀₀ₐ-L_{100f}. The plurality of sub-lengths L₁₀₀ₐ-L_{100f} include a first sub-length L₁₀₀ₐ, a second sub-length L_{100b}, a third sub-length L_{100c}, a fourth sub-length L_{100d}, a fifth sub-length L₁₀₀ₑ, and a sixth sub-length L_{100f}.

The first sub-length L₁₀₀ₐ of the length L₁₀₀ of the cannula carrier 100 defines the length of the neck portion 102a of the substantially tube-shaped body 102. The first sub-length L₁₀₀ₐ extends between the first proximal end surface portion 104a of the proximal end surface 104 of the substantially tube-shaped body 102 and the second shoulder surface portion 108c₂ of the third outer side surface portion 108c of the outer side surface 108 of the substantially tube-shaped body 102.

The second sub-length L_{100b} of the length L₁₀₀ of the cannula carrier 100 defines the length of the head portion 102b of the substantially tube-shaped body 102. The second sub-length L_{100b} extends between the second shoulder surface portion 108c₂ of the third outer side surface portion 108c of the outer side surface 108 of the substantially tube-shaped body 102 and the distal-most portion of the third distal end surface portion 106c of the distal end surface 106 of the substantially tube-shaped body 102.

The third sub-length L_{100c} of the length L₁₀₀ of the cannula carrier 100 defines the length of the second proximal end surface portion 104b of the proximal end surface 104 of the substantially tube-shaped body 102. The third sub-length L_{100c} extends between the first proximal end surface portion 104a of the proximal end surface 104 of the substantially tube-shaped body 102 and a proximal-most end of the first outer side surface portion 108a of the outer side surface 108 of the substantially tube-shaped body 102.

The fourth sub-length L_{100d} of the length L₁₀₀ of the cannula carrier 100 defines the length of the dome-shaped portion of the head portion 102b of the substantially tube-shaped body 102 defined by the third distal end surface portion 106c distal end surface 106 of the substantially tube-shaped body 102. The fourth sub-length L_{100d} extends between a distal-most end of the second outer side surface portion 108b of the outer side surface 108 of the substantially tube-shaped body 102 and the distal-most end of the third distal end surface portion 106c distal end surface 106 of the substantially tube-shaped body 102.

The fifth sub-length L₁₀₀ₑ of the length L₁₀₀ of the cannula carrier 100 defines the length, radial distal, or the depth that the first shoulder surface portion 108c₁ extends into the third outer side surface portion 108c of the outer side surface 108 of the substantially tube-shaped body 102. The fifth sub-length L₁₀₀ₑ extends between the second shoulder surface portion 108c₂ of the third outer side surface portion 108c of the outer side surface 108 of the substantially tube-shaped body 102 and the distal-most portion or the deepest-most portion of the arcuate recess defined by the first shoulder surface portion 108c₁ of the third outer side surface portion 108c of the outer side surface 108 of the substantially tube-shaped body 102.

The sixth sub-length L_{100f} of the length L₁₀₀ of the cannula carrier 100 defines the length or axial depth of the centrally recessed circular well defined by the first distal end surface portion 106a and the second distal end surface portion 106b of the third distal end surface portion 106c of the distal end surface 106 of the substantially tube-shaped body 102. The sixth sub-length L_{100f} extends between the first distal end surface portion 106a of the distal end surface 106 of the substantially tube-shaped body 102 and the distal-most portion of the second distal end surface portion 106b of the distal end surface 106 of the substantially tube-shaped body 102.

Referring to FIGS. 19A-19E, a method for assembling the hypodermic interface assembly 10 is described. Firstly, at FIG. 19A, some of the components (i.e., the hub 14, the cannula carrier 100, and a fastening portion 200a of the one or more fastening portions 200) of the hypodermic interface assembly 10 are axially aligned about a central axis A₁₀-A₁₀ (see also FIG. 1). The central axis A₁₀-A₁₀ corresponds to, for example, the central axes A₁₄-A₁₄, A₁₀₀-A₁₀₀ of each of the hub 14 and the cannula carrier 100 of the one or more fastening portions 200.

As will be described in the following disclosure at FIGS. 19A-19E, the one or more fastening portions 200 may be an adhesive material. Although the one or more fastening portions 200 are shown at FIGS. 19A-19E for connecting the cannula 12, the hub 14, and the cannula carrier 100, other implementations for forming the hypodermic interface assembly 10 may not utilize one or more of the one or more fastening portions 200 (see, e.g., the hypodermic interface assemblies 10′, 10″ at FIGS. 26-28 and 29-31). If the one or more fastening portions 200 are utilized in the design of any of the hypodermic interface assemblies 10′, 10″, exemplary adhesive materials may include, for example: an acrylic adhesive, a cyanoacrylate adhesive, a ultra-violet (UV) curable adhesive, or the like.

In some instances, the selected type of adhesive of the one or more fastening portions 200 may be selected in order to provide reliable adhesion between the hub 14, which may be formed from a metal material, and the cannula carrier 100, which may be formed from a plastic material. In other examples, the selected type of adhesive of the one or more fastening portions 200 may be selected in order to provide reliable adhesion between the cannula 12, which may a metal material coated with silicon oil, and the cannula carrier 100, which may be formed from a plastic material.

With reference to FIG. 19A, the proximal end surface 104 of the substantially tube-shaped body 102 of the cannula carrier 100 is arranged opposite the distal opening 48 of the hub 14 that is defined by the distal end surface 38 of the substantially tube-shaped body 34 of the hub 14. As seen at FIG. 19A and as described above, the proximal end surface 104 of the substantially tube-shaped body 102 of the cannula carrier 100 and the first outer side surface portion 108a of the outer side surface 108 of the substantially tube-shaped body 102 of the cannula carrier 100 generally define the neck portion 102a of the substantially tube-shaped body 102 of the cannula carrier 100.

As described above at FIG. 18, the first outer side surface portion 108a of the substantially tube-shaped body 102 of the cannula carrier 100 extends away from the central axis A₁₀₀-A₁₀₀ of the cannula carrier 100 at the first radial distance R₁₀₈ₐ thereby defining the first outer diameter D₁₀₂ₐ of the substantially tube-shaped body 102, which may be alternatively referred to as a largest diameter of the neck portion 102a of the substantially tube-shaped body 102 of the cannula carrier 100 (whereas the proximal end surface 104 of the substantially tube-shaped body 102 of the cannula carrier 100 defines a substantially cone-shaped portion of the neck portion 102 of the substantially tube-shaped body 102 of the cannula carrier 100 that progressively decreases in diameter as the second proximal end surface portion 104b extends from the first outer side surface portion 108a to the first proximal end surface portion 104a). The largest diameter D₁₀₂ₐ of the neck portion 102a of the substantially tube-shaped body 102 of the cannula carrier 100 may be approximately equal to but slightly less than the distal opening diameter D₄₈ (see, e.g., FIG. 7) of the distal opening 48 formed by the distal end surface 38 of the substantially tube-shaped body 34 of the hub 14 as well as the passage diameter D₄₄ (see, e.g., FIG. 7) defined by the inner surface 42 of the passage 44 formed by the substantially tube-shaped body 34 of the hub 14. Accordingly, the distal opening 48 formed by the distal end surface 38 of the substantially tube-shaped body 34 of the hub 14 is sized for permitting passage of the neck portion 102a of the substantially tube-shaped body 102 of the cannula carrier 100 there-through. Furthermore, the passage 44 formed by the substantially tube-shaped body 34 of the hub 14 is sized for receiving and containing the neck portion 102a of the substantially tube-shaped body 102 of the cannula carrier 100.

As seen at FIG. 19A, a first step in the process for assembling of the hypodermic interface assembly 10 includes arranging the fastening portion 200a upon the outer side surface 108 of the substantially tube-shaped body 102 of the cannula carrier 100. In some implementations, the fastening portion 200a may be arranged upon the first outer side surface portion 108a of the outer side surface 108 of the substantially tube-shaped body 102 of the cannula carrier 100. In some instances, the fastening portion 200a may be arranged upon the first outer side surface portion 108a of the outer side surface 108 near the second proximal end surface portion 104b of the proximal end surface 104 of the substantially tube-shaped body 102 of the cannula carrier 100.

In some configurations, the fastening portion 200a may an adhesive, glue, or the like. In some instances, the selected type of adhesive of fastening portion 200a of the one or more fastening portions 200 may be selected in order to provide reliable adhesion between the hub 14, which may be formed from a metal material, and the cannula carrier 100, which may be formed from a plastic material. In some instances, the fastening portion 200a of the one or more fastening portions 200 may include, for example: an acrylic adhesive; a cyanoacrylate adhesive; or the like. In some examples, the fastening portion 200a of the one or more fastening portions 200 may be an epoxy resin. In other examples, the fastening portion 200a of the one or more fastening portions 200 may be a ultra-violet (UV) curable epoxy resin.

As seen at FIGS. 1 and 19A, the fastening portion 200a may be applied to the first outer side surface portion 108a of the outer side surface 108 of the substantially tube-shaped body 102 of the cannula carrier 100 in the form of an uninterrupted ring bead of adhesive. Although the fastening portion 200a may be arranged upon the first outer side surface portion 108a of the outer side surface 108 of the substantially tube-shaped body 102 of the cannula carrier 100 in the form of an uninterrupted ring bead of adhesive, the fastening portion 200a may be arranged upon the first outer side surface portion 108a of the outer side surface 108 of the substantially tube-shaped body 102 of the cannula carrier 100 in other configurations such as, for example, in the form of a series of adhesive bead dollops that are arranged, for example in series, forming a circumferential ring configuration. Because the fastening portion 200a, which may be an adhesive in some configurations, is applied in the form or a ring bead or a plurality if bead dollops (i.e., the adhesive 200a is not initially applied upon most of or entirely upon the first outer side surface portion 108a of the outer side surface 108 of the substantially tube-shaped body 102 of the cannula carrier 100 as seen at FIG. 19A), the adhesive 200a is applied in a minimalistic fashion, reducing the amount of adhesive utilized in the design of the hypodermic interface assembly 10, thereby reducing the cost and weight of the hypodermic interface assembly 10.

Referring to FIGS. 19A and 19B, in some implementations, when the fastening portion 200a is an adhesive, prior to the adhesive 200a curing and solidifying, the neck portion 102a of the substantially tube-shaped body 102 of the cannula carrier 100 is inserted through the distal opening 48 formed by the distal end surface 38 of the substantially tube-shaped body 34 of the hub 14. As described above, the second proximal end surface portion 104b extends at the angle θ_{104b} (see, e.g., FIGS. 12, 13, 17, and 18) from the first proximal end surface portion 104a for defining a substantially conical portion of the proximal end surface 104; the conical portion of the proximal end surface 104 may assisting in locating or guiding the proximal end surface 104 of the cannula carrier 100 through the distal opening 48 formed by the distal end surface 38 of the substantially tube-shaped body 34 of the hub 14.

As the neck portion 102a of the substantially tube-shaped body 102 of the cannula carrier 100 is disposed into the passage 44 formed by the substantially tube-shaped body 34 of the hub 14 according to the direction of the arrow Y, the distal end surface 38 of the substantially tube-shaped body 34 of the hub 14 axially comes into contact with the adhesive 200a, and, as a result, the distal end surface 38 of the substantially tube-shaped body 34 of the hub 14 axially pushes, spreads, or wipes the adhesive 200a along the first outer side surface portion 108a of the outer side surface 108 of the substantially tube-shaped body 102 of the cannula carrier 100 in a direction according to the arrow Y′ (which is opposite the direction of the arrow Y) toward the third outer side surface portion 108c of the outer side surface 108 of the substantially tube-shaped body 102 of the cannula carrier 100.

As seen at FIGS. 19B and 19C, because the distal end surface 38 of the substantially tube-shaped body 34 of the hub 14 axially pushes, spreads, or wipes (according to the arrow Y′) the adhesive 200a along the first outer side surface portion 108a of the outer side surface 108 of the substantially tube-shaped body 102 of the cannula carrier 100 in a direction according to the arrow Y′ toward the third outer side surface portion 108c of the outer side surface 108 of the substantially tube-shaped body 102 of the cannula carrier 100, the adhesive 200a may be arranged over most or substantially all of the first outer side surface portion 108a of the outer side surface 108 of the substantially tube-shaped body 102 of the cannula carrier 100. As seen at FIG. 19C, none of the adhesive 200a initially was arranged upon the proximal end surface 104 of the substantially tube-shaped body 102 of the cannula carrier 100 and, in some configurations, none of the adhesive 200a is transferred to the proximal end surface 104 of the substantially tube-shaped body 102 of the cannula carrier 100 due to the fact that the distal end surface 38 of the substantially tube-shaped body 34 of the hub 14 axially pushes, spreads, or wipes the adhesive 200a along the first outer side surface portion 108a of the outer side surface 108 of the substantially tube-shaped body 102 of the cannula carrier 100 in a direction (according to the arrow Y′) away from the proximal end surface 104 of the substantially tube-shaped body 102 of the cannula carrier 100 and toward the third outer side surface portion 108c of the outer side surface 108 of the substantially tube-shaped body 102 of the cannula carrier 100.

Furthermore, as seen at FIG. 19C, because the distal end surface 38 of the substantially tube-shaped body 34 of the hub 14 axially pushes, spreads, or wipes the adhesive 200a along the first outer side surface portion 108a of the outer side surface 108 of the substantially tube-shaped body 102 of the cannula carrier 100 in a direction (according to the arrow Y′) toward the third outer side surface portion 108c of the outer side surface 108 of the substantially tube-shaped body 102 of the cannula carrier 100, the adhesive 200a may ultimately, substantially or in part, fill the arcuate recess defined by the first shoulder surface portion 108c₁ of the third outer side surface portion 108c of the outer side surface 108 of the substantially tube-shaped body 102 of the cannula carrier 100 (i.e., the first shoulder surface portion 108c₁ of the third outer side surface portion 108c may be specifically configured to receive adhesive 200a in order to mitigate excess adhesive being radially squeezed out of a gap defined by opposing surfaces defined by the third outer side surface portion 108c of the outer side surface 108 of the substantially tube-shaped body 102 of the cannula carrier 100 and the distal end surface 38 of the substantially tube-shaped body 34 of the hub 14). Yet even further, in some configurations, should the adhesive 200a completely fill the arcuate recess defined by the first shoulder surface portion 108c₁ of the third outer side surface portion 108c of the outer side surface 108 of the substantially tube-shaped body 102 of the cannula carrier 100, once the distal end surface 38 of the substantially tube-shaped body 34 of the hub 14 is arranged adjacent the second shoulder surface portion 108c₂ of the third outer side surface portion 108c of the outer side surface 108 of the substantially tube-shaped body 102 of the cannula carrier 100 as a result of fully disposing the neck portion 102a of the substantially tube-shaped body 102 of the cannula carrier 100 within the passage 44 formed by the substantially tube-shaped body 34 of the hub 14, the adhesive 200a may overflow out of the arcuate recess defined by the first shoulder surface portion 108c₁ of the third outer side surface portion 108c of the outer side surface 108 of the substantially tube-shaped body 102 of the cannula carrier 100 and flow radially outwardly away from the central axis A₁₀-A₁₀ of the hypodermic interface assembly 10 such that the adhesive 200a may be arranged upon the second shoulder surface portion 108c₂ of the third outer side surface portion 108c of the outer side surface 108 of the substantially tube-shaped body 102 of the cannula carrier 100 for joining the second shoulder surface portion 108c₂ of the third outer side surface portion 108c of the outer side surface 108 of the substantially tube-shaped body 102 of the cannula carrier 100 to the distal end surface 38 of the substantially tube-shaped body 34 of the hub 14.

Referring to FIG. 19C, in some configurations, the adhesive 200a is arranged upon most of or all of the first outer side surface portion 108a and the third outer side surface portion 108c of the outer side surface 108 of the substantially tube-shaped body 102 of the cannula carrier 100. Accordingly, the adhesive 200a substantially fills a narrow gap between: (1) the first outer side surface portion 108a; and (2) the third outer side surface portion 108c of the outer side surface 108 of the substantially tube-shaped body 102 of the cannula carrier 100 that are arranged, respectively, opposite: (1) the inner surface 42 of the substantially tube-shaped body 34 of the hub 14; and (2) the distal end surface 38 of the substantially tube-shaped body 34 of the hub 14 in order to axially and radially adhesively connect the cannula carrier 100 to the hub 14 for non-removably-securing the cannula carrier 100 to the hub 14.

With reference back to FIG. 19A, although the fastening portion 200a is shown applied to the first outer side surface portion 108a of the outer side surface 108 of the substantially tube-shaped body 102 of the cannula carrier 100, the fastening portion 200a may alternatively or also applied to the inner surface 42 of the substantially tube-shaped body 34 of the hub 14 that defines the passage 44 formed by the substantially tube-shaped body 34 of the hub 14. In some configurations, if the fastening portion 200a is alternatively applied or also applied to the inner surface 42 of the substantially tube-shaped body 34 of the hub 14, the fastening portion 200a may be applied to the inner surface 42 of the substantially tube-shaped body 34 of the hub 14 near the distal end surface 38 of the substantially tube-shaped body 34 of the hub 14. Therefore, as the neck portion 102a of the substantially tube-shaped body 102 of the cannula carrier 100 is disposed into the passage 44 formed by the substantially tube-shaped body 34 of the hub 14 according to the direction of the arrow Y, the neck portion 102a may move through the adhesive 200a, which may be in the form of a ring bead, in order to axially spread or wipe the adhesive 200a along the first outer side surface portion 108a of the outer side surface 108 of the substantially tube-shaped body 102 of the cannula carrier 100 in the same direction according to the arrow Y. In other configurations, the adhesive 200a may be applied to one or both of the third outer side surface portion 108c of the outer side surface 108 of the substantially tube-shaped body 102 of the cannula carrier 100 and the distal end surface 38 of the substantially tube-shaped body 34 of the hub 14 in order to “sandwich” the adhesive 200a between the third outer side surface portion 108c of the outer side surface 108 of the substantially tube-shaped body 102 of the cannula carrier 100 and the distal end surface 38 of the substantially tube-shaped body 34 of the hub 14; in such a configuration, the adhesive 200a may or may not be arranged upon the first outer side surface portion 108a of the outer side surface 108 of the substantially tube-shaped body 102 of the cannula carrier 100.

With reference to FIG. 19C, once the adhesive 200a is pushed, spread, wiped, or otherwise arranged as desired upon one or both of the hub 14 and the cannula carrier 100 as a result of disposing the neck portion 102a of the substantially tube-shaped body 102 of the cannula carrier 100 into the passage 44 formed by the substantially tube-shaped body 34 of the hub 14 according to the direction of the arrow Y, the adhesive 200a may be permitted to cure or solidify for non-removably-securing the cannula carrier 100 to the hub 14. In some instances, a period of curing time may be required in order to permit the adhesive 200a to cure or solidify. In other configurations, if the adhesive 200a is an UV curable epoxy resin, the subassembly defined by the hub 14, the cannula carrier 100, and the UV curable epoxy resin 200a may be exposed to an UV light source (not shown) that activates the UV curable epoxy resin 200a in order to cure or solidify the UV curable epoxy resin 200a.

As seen at FIG. 19C, a portion of the cannula 12 including the proximal end surface 18 at the proximal end 16_{P} of the tube-shaped body 16 is shown arranged near the subassembly of the hypodermic interface assembly 10 that includes the hub 14, the cannula carrier 100, and the cured or solidified adhesive 200a. The central axis A₁₂-A₁₂ (see, e.g., FIG. 2) of the cannula 12 is axially aligned with the central axes A₁₄-A₁₄ and A₁₀₀-A₁₀₀, of each of the hub 14 and the cannula carrier 100. The central axes A₁₂-A₁₂, A₁₄-A₁₄, and A₁₀₀-A₁₀₀, of each of the cannula 12, the hub 14, and the cannula carrier 100 correspond to the central axis A₁₀-A₁₀ (see FIG. 1) of the hypodermic interface assembly 10.

As seen at FIG. 19E and as described above, the outer surface 22 of the tube-shaped body 16 of the cannula 12 defines an outer diameter D₁₂ of the cannula 12. The outer diameter D₁₂ of the cannula 12 is less than the distal opening diameter D₁₁₆ (see, e.g., FIG. 18) defined by the distal opening 116 formed by the distal end surface 106 of the substantially tube-shaped body 102 of the cannula carrier 100. Furthermore, the outer diameter D₁₂ of the cannula 12 may be approximately equal to but slightly less than the intermediate opening diameter D₁₁₅ (see, e.g., FIG. 118) defined by the intermediate opening 115 formed by the distal end surface portion 106a of the distal end surface 106 of the substantially tube-shaped body 102 of the cannula carrier 100. Accordingly, when the cannula 12 is joined to the cannula carrier 100 by inserting the cannula 12 into the openings 115, 116 according to the direction of the arrow Y (FIG. 19C), the distal opening 116 formed by the distal end surface 106 of the substantially tube-shaped body 102 of the cannula carrier 100 is sized for permitting passage of the proximal end 16_{P} of the tube-shaped body 16 of the cannula 12 there-through; furthermore when the cannula 12 is joined to the cannula carrier 100, the intermediate opening 115 formed by the distal end surface portion 106a of the distal end surface 106 of the substantially tube-shaped body 102 of the cannula carrier 100 is sized for permitting passage of the proximal end 16_{P} of the tube-shaped body 16 of the cannula 12 there-through. Furthermore, the outer diameter D₁₂ of the cannula 12 may be approximately equal to but slightly less than first passage diameter D₁₁₂ₐ of the first passage portion 112a of the passage 112 formed by the substantially tube-shaped body 102 of the cannula carrier 100; accordingly, the first passage portion 112a of the passage 112 formed by the substantially tube-shaped body 102 of the cannula carrier 100 is sized for receiving and containing a portion of the length L₁₂ (e.g., the sub-length portion L_{12b1} as seen at FIG. 2) of the cannula 12.

With reference to FIG. 19D, the cannula 12 may be initially joined to the cannula carrier 100 by friction-fit or a combination of friction fit and adhesive securing a portion of the outer surface 22 of the tube-shaped body 16 of the cannula 12 to the inner surface 110 of the substantially tube-shaped body 102 of the cannula carrier 100 that defines the first passage portion 112a of the passage 112 extending through the substantially tube-shaped body 102 of the cannula carrier 100. As seen at FIG. 19D, the cannula 12 may be passed through the first passage portion 112a of the passage 112 such that the proximal end 16_{P} of the tube-shaped body 16 of the cannula 12 is arranged axially beyond the first proximal end surface portion 104a of the proximal end surface 104 of the substantially tube-shaped body 102 of the cannula carrier 100. In an exemplary configuration as see at FIG. 19D, a proximal portion of the length L₁₂ (i.e., the sub-length portion L₁₂ₐ₁ and the sub-length portion L_{12b2} as seen at FIG. 2) is arranged beyond the first proximal end surface portion 104a of the proximal end surface 104 of the substantially tube-shaped body 102 of the cannula carrier 100 and a distal portion of the length L₁₂ (i.e., the sub-length portion L₁₂ₐ₂ as seen at FIG. 2) is arranged beyond the distal-most portion of the third distal end surface portion 106c of the distal end surface 106 of the substantially tube-shaped body 102 of the cannula carrier 100 while an intermediate portion of the length L₁₂ (i.e., the sub-length portion L_{12b1} and the sub-length portion L_{12b3} as seen at FIG. 2) is disposed within, contained by the passage 112 extending through the substantially tube-shaped body 102 of the cannula carrier 100 for friction-fit connecting the outer surface 22 of the tube-shaped body 16 of cannula 12 to the inner surface 110 of the substantially tube-shaped body 102 of hub 14. Optionally, the friction-fit connecting the outer surface 22 with the tube-shaped body 16 of cannula 12 to the inner surface 110 of the substantially tube-shaped body 102 of hub 14 may be further strengthened using an adhesive.

As seen at FIGS. 1 and 19D, a fastening portion 200b of the one or more fastening portions 200) of the hypodermic interface assembly 10 is axially aligned about the central axis A₁₀-A₁₀ of the hypodermic interface assembly 10. As similarly discussed above, the central axes A₁₂-A₁₂, A₁₄-A₁₄, and A₁₀₀-A₁₀₀, of each of the cannula 12, the hub 14, and the cannula carrier 100 correspond to the central axis A₁₀-A₁₀ of the hypodermic interface assembly 10.

With reference to FIG. 19D, the fastening portion 200b is defined by a thickness T_{200b} extending between a proximal surface of the fastening portion 200b and a distal surface of the fastening portion 200b. The fastening portion 200b is also defined by an inner surface that defines a passage diameter D_{200bP} of the fastening portion 200b. The fastening portion 200b is also defined by an outer surface that defines an outer diameter D_{200bO} of the fastening portion 200b.

Although the fastening portion 200b is shown and described at FIGS. 1 and 19D to be a pre-formed ring of material having proximal, distal, inner, and outer surfaces, the fastening portion 200b may be a non-solid or liquid material with undefined surfaces until the non-solid or liquid material is poured, extruded into, and settles within the second passage portion 112b of the passage 112 of the cannula carrier 100 (and after the cannula 12 is disposed within the first passage portion 112a of the passage 112 of the cannula carrier 100 as described above at FIGS. 19C and 19D). If the fastening portion 200b is a non-solid or liquid material that is poured or extruded into the second passage portion 112b of the passage 112 of the cannula carrier 100, a final shape of the fastening portion 200b may resemble a ring-shape (as seen at FIGS. 1 and 19D) as a result of the shape of the outer diameter D₁₂ of the cannula 12 (after the cannula 12 is disposed within the first passage portion 112a of the passage 112 of the cannula carrier 100 as described above at FIGS. 19C and 19D) and the second passage diameter D_{112b} of the second passage portion 112b of the passage 112 of the cannula carrier 100. Accordingly, although the fastening portion 200b may be a non-solid or liquid material that is poured or extruded into the second passage portion 112b of the passage 112 of the cannula carrier 100, the fastening portion 200b is described below in the context of a pre-formed or solid material in order to identify structural surfaces and geometric configurations of the fastening portion 200b relative surfaces and geometric configurations of the cannula 12 and the cannula carrier 100.

The outer diameter D₁₂ of the cannula 12 may be approximately equal to but slightly less than the passage diameter D_{200bP} defined by the inner surface of the fastening portion 200b. Accordingly, as seen at FIG. 19E, the outer diameter D₁₂ defined by the outer surface 22 of the tube-shaped body 16 of the cannula 12 is sized for being received and contained within the passage diameter D_{200bP} defined by the inner surface of the fastening portion 200b.

Furthermore, the outer diameter D_{200bO} defined by the outer surface of the fastening portion 200b may be approximately equal to but slightly less than: (1) the distal opening diameter D₁₁₆ defined by the distal opening 116 formed by the distal end surface 106 of the substantially tube-shaped body 102 of the cannula carrier 100; and (2) the second passage diameter D_{112b} of the second passage portion 112b of the passage 112. Accordingly, as seen at FIG. 19E, the outer diameter D_{200bO} defined by the outer surface of the fastening portion 200b is sized for being received and contained within the second passage diameter D_{112b} of the second passage portion 112b of the passage 112.

With reference to FIG. 19E, when the fastening portion 200b is arranged within the second passage portion 112b of the passage 112, the proximal surface of the fastening portion 200b may be disposed adjacent the distal end surface portion 106a of the distal end surface 106 of the substantially tube-shaped body 102 of the cannula carrier 100, and, the outer side surface of the fastening portion 200b may be disposed adjacent the second distal end surface portion 106b of the distal end surface 106 of the substantially tube-shaped body 102. Furthermore, the distal surface of the fastening portion 200b may be aligned or substantially co-planar with the distal-most portion of the third distal end surface portion 106c of the distal end surface 106 of the substantially tube-shaped body 102 of the cannula carrier 100. Furthermore, the thickness T_{200b} extending between the proximal surface and the distal surface of the fastening portion 200b may be substantially equal to the sub-length portion L_{12b3} (see, e.g., FIG. 2) of the length L₁₂ of the cannula 12; accordingly, the fastening portion 200b may be configured to surround and be disposed adjacent the outer surface 22 of the tube-shaped body 16 of the cannula 12 defined by the sub-length portion L_{12b3} of the length L₁₂ of the cannula 12 for adhesively-securing the cannula 12 to the distal end 102_{D} of the cannula carrier 100.

In some configurations, the fastening portion 200b may an adhesive, glue, or the like. In some instances, the selected type of adhesive of fastening portion 200b of the one or more fastening portions 200 may be selected in order to provide reliable adhesion between the cannula 12, which may a metal material coated with silicon oil, and the cannula carrier 100, which may be formed from a plastic material.

In some examples, the fastening portion 200b may be an epoxy resin. In other examples, the fastening portion 200b may be an UV curable epoxy resin. If the fastening portion 200b, which may be an adhesive, is an UV curable epoxy resin, the UV curable epoxy resin 200b may be exposed to an UV light source (not shown) that activates the UV curable epoxy resin 200b in order to cure or solidify the UV curable epoxy resin 200b for non-removably-securing a portion of the outer surface 22 of the tube-shaped body 16 of the cannula 12 defined by sub-length portion L_{12b3} (see, e.g., FIG. 2) of the length L₁₂ of the cannula 12 to the first distal end surface portion 106a and the second distal end surface portion 106b of the distal end surface 106 of the substantially tube-shaped body 102 of the cannula carrier 100 that defines the second passage portion 112b of the passage 112 of the cannula carrier 100. Once cured, the fastening portion 200b axially secures the cannula 12 to the distal end 102_{D} of the cannula carrier 100 in order to mitigate axial removal or extraction of the cannula 12 from the distal end 102_{D} of the cannula carrier 100.

As seen at FIGS. 1 and 19D, a fastening portion 200c of the one or more fastening portions 200) of the hypodermic interface assembly 10 is axially aligned about the central axis A₁₀-A₁₀ (see also FIG. 1). As similarly discussed above, the central axes A₁₂-A₁₂, A₁₄-A₁₄, and A₁₀₀-A₁₀₀ of each of the cannula 12, the hub 14, and the cannula carrier 100 correspond to the central axis A₁₀-A₁₀ of the hypodermic interface assembly 10.

With reference to FIG. 19D, the fastening portion 200c is defined by a thickness T_{200c} extending between a proximal surface of the fastening portion 200c and a distal surface of the fastening portion 200c. The fastening portion 200c is also defined by an inner surface that defines a passage diameter D_{200cP} of the fastening portion 200c. The fastening portion 200c is also defined by an outer surface that defines an outer diameter D_{200cO} of the fastening portion 200c.

Although the fastening portion 200c is shown and described at FIGS. 1 and 19D to be a pre-formed ring of material having proximal, distal, inner, and outer surfaces, the fastening portion 200c may be a non-solid or liquid material with undefined surfaces until the non-solid or liquid material is extruded: (1) around a portion of the length L₁₂ (i.e., the sub-length portion L_{12b2}) of the cannula 12 defined by the outer surface 22 of the tube-shaped body 16 of the cannula 12; and (2) adjacent the first proximal end surface portion 104a of the proximal end surface 104 of the substantially tube-shaped body 102 of the cannula carrier 100 that surrounds the proximal opening 114 formed by the proximal end surface 104 of the substantially tube-shaped body 102 of the cannula carrier 100 (after the cannula 12 is disposed within the first passage portion 112a of the passage 112 of the cannula carrier 100 as described above at FIGS. 19C and 19D). If the fastening portion 200c is a non-solid or liquid material that is extruded as described above, a final shape of the fastening portion 200c may resemble a ring-shape (as seen at FIGS. 1 and 19D) as a result of the shape of the outer diameter D₁₂ of the cannula 12 (after the cannula 12 is disposed within the first passage portion 112a of the passage 112 of the cannula carrier 100 as described above at FIGS. 19C and 19D). Accordingly, although the fastening portion 200c may be a non-solid or liquid material that is poured or extruded into the second passage portion 112b of the passage 112 of the cannula carrier 100, the fastening portion 200c is described below in the context of a pre-formed or solid material in order to identify structural surfaces and geometric configurations of the fastening portion 200c relative surfaces and geometric configurations of the cannula 12 and the cannula carrier 100.

The outer diameter D₁₂ of the cannula 12 may be approximately equal to but slightly less than the passage diameter D_{200cP} defined by the inner surface of the fastening portion 200c. Accordingly, as seen at FIG. 19E, the outer diameter D₁₂ defined by the outer surface 22 of the tube-shaped body 16 of the cannula 12 is sized for being received and contained within the passage diameter D_{200cP} defined by the inner surface of the fastening portion 200c.

With reference to FIG. 19E, the fastening portion 200c is arranged: (1) around the portion of the length L₁₂ (i.e., the sub-length portion L_{12b2}) of the cannula 12 defined by the outer surface 22 of the tube-shaped body 16 of the cannula 12; and (2) adjacent the first proximal end surface portion 104a of the proximal end surface 104 of the substantially tube-shaped body 102 of the cannula carrier 100 that surrounds the proximal opening 114 formed by the proximal end surface 104 of the substantially tube-shaped body 102 of the cannula carrier 100. Accordingly, the thickness T_{200c} extending between the proximal surface and the distal surface of the fastening portion 200c is substantially equal to the sub-length portion L_{12b2} of the length L₁₂ of the cannula 12. As such, the fastening portion 200c is configured to surround and be disposed adjacent the outer surface 22 of the tube-shaped body 16 of the cannula 12 defined by the sub-length portion L_{12b2} of the length L₁₂ of the cannula 12 for adhesively connecting the proximal end 102_{P} of the cannula carrier 100 to the cannula 12.

In some configurations, the fastening portion 200c may an adhesive, glue, or the like. In some instances, the selected type of adhesive of fastening portion 200c of the one or more fastening portions 200 may be selected in order to provide reliable adhesion between the cannula 12, which may a metal material coated with silicon oil, and the cannula carrier 100, which may be formed from a plastic material.

In some examples, the fastening portion 200c may be an epoxy resin. In other examples, the fastening portion 200c may be an UV curable epoxy resin. In other examples, the fastening portion 200c may be a cyanoacrylate adhesive, a polyester resin, or a urethane. If the fastening portion 200c, which may be an adhesive, is an UV curable epoxy resin, the UV curable epoxy resin 200c may be exposed to an UV light source (not shown) that activates the UV curable epoxy resin 200c in order to cure or solidify the UV curable epoxy resin 200c for non-removably-securing a portion of the outer surface 22 of the tube-shaped body 16 of the cannula 12 defined by sub-length portion L_{12b2} of the length L₁₂ of the cannula 12 to a portion of the first proximal end surface portion 104a of the proximal end surface 104 of the substantially tube-shaped body 102 of the cannula carrier 100 that surrounds the proximal opening 114 formed by the proximal end surface 104 of the substantially tube-shaped body 102 of the cannula carrier 100. Once cured, the fastening portion 200c axially secures the cannula 12 to the proximal end 102_{P} of the cannula carrier 100 in order to mitigate axial removal or extraction of the cannula 12 from the proximal end 102_{P} of the cannula carrier 100.

As described above, although the hypodermic interface assembly 10 has been described to include a first fastening portion 200a, a second fastening portion 200b, and a third fastening portion 200c, exemplary implementations of the hypodermic interface assembly 10 may be practiced using one, two, or even none of the first, second, and third fastening portions 200a, 200b, 200c of the one or more fastening portions 200. In some configurations when less than three of the first fastening portion 200a, the second fastening portion 200b, and the third fastening portion 200c of the one or more fastening portions 200 are utilized in an exemplary configuration of the hypodermic interface assembly 10, sub-assemblies including two or more of the cannula 12, the hub 14, and the cannula carrier 100 may be joined in any desirable manner such as, for example, a friction-fit configuration, a mechanical configuration (e.g., a threaded configuration), an interference-fit connection, or the like. Optionally, the friction-fit connecting the outer surface 22 with the tube-shaped body 16 of cannula 12 to the inner surface 110 of the substantially tube-shaped body 102 of hub 14 may be further strengthened using an adhesive.

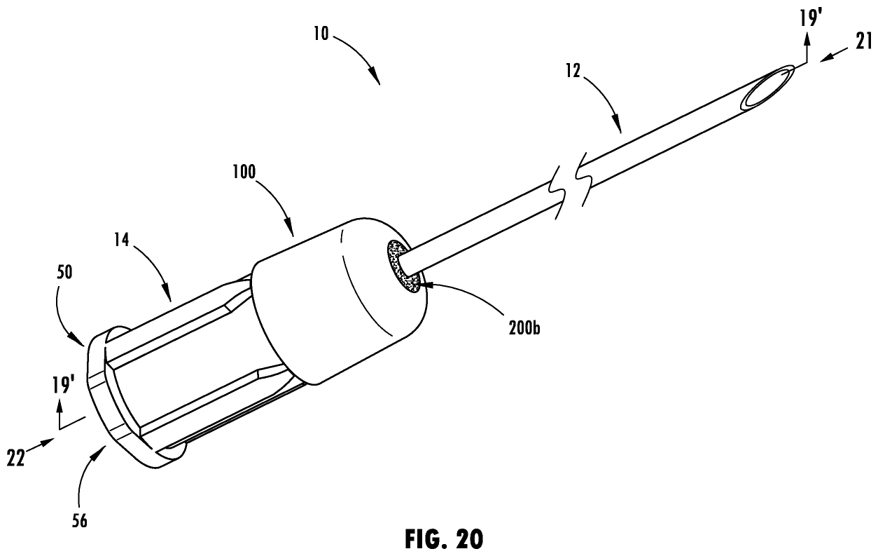

Referring to FIGS. 20-25, a plurality of views of the hypodermic interface assembly 10 arranged in an assembled state are shown. The exemplary configuration of the hypodermic interface assembly 10 at FIGS. 20-25 include, for example: the cannula 12; the hub 14; the cannula carrier 100; and, optionally, the one or more fastening portions 200 including the first fastening portion 200a, the second fastening portion 200b, and the third fastening portion 200c. Although the one or more fastening portions 200 including the first fastening portion 200a, the second fastening portion 200b, and the third fastening portion 200c are represented in the exemplary configuration of the hypodermic interface assembly 10 at FIGS. 20-25, as described above, the one or more fastening portions 200 may be optional; as such, with reference to FIGS. 26-28 and 29-31, as examples, two or more of the cannula 12, the hub 14, and the cannula carrier 100 may be joined in any desirable manner such as, for example, a friction-fit configuration, a mechanical configuration (e.g., a threaded configuration), an interference-fit connection, or the like without utilizing one or more of the first fastening portion 200a, the second fastening portion 200b, and the third fastening portion 200c.

Firstly, with reference to FIGS. 26-28, another hypodermic interface assembly is shown generally at 10′. The hypodermic interface assembly 10′ is substantially similar to the design of the hypodermic interface assembly 10 with the exception that the hypodermic interface assembly 10′ omits the third fastening portion 200c. Because the hypodermic interface assembly 10′ omits the third fastening portion 200c, the tube-shaped body 16′ of the cannula 12′ may include, for example, a collar 200c′ that projects radially outwardly from the tube-shaped body 16′ of the cannula 12′ near the proximal end 16_{P}′ of the tube-shaped body 16′ of the cannula 12′.

In such a configuration, with reference to FIG. 26, a sub-assembly of the hypodermic interface assembly 10′ including the cannula 12′ and the cannula carrier 100′ may be firstly formed by inserting the distal end 16_{D}′ of the tube-shaped body 16′ of the cannula 12′ through the proximal opening 114′ formed by the proximal end surface 104′ of the substantially tube-shaped body 102′ of the cannula carrier 100′ according to the direction of the arrow Y′. The collar 200c′ may define a diameter that is larger than the proximal opening diameter D₁₁₄′ defined by the proximal opening 114′ formed by the proximal end surface 104′ of the substantially tube-shaped body 102′ of the cannula carrier 100′. Accordingly, a distal surface of the collar 200c′ may be disposed adjacent the first proximal end surface portion 104a′ of the proximal end surface 104′ of the substantially tube-shaped body 102′ of the cannula carrier 100′; therefore, because the diameter of the collar 200c′ is larger than the proximal opening diameter D₁₁₄ defined by the proximal opening 114′ formed by the proximal end surface 104′ of the substantially tube-shaped body 102′ of the cannula carrier 100′, the cannula 12′ may be prevented from being axially pulled through the passage 112′ of the cannula carrier 100′ and out of the distal end 102_{D}′ of the cannula carrier 100′.

In the exemplary configuration of the hypodermic interface assembly 10′, the first fastening portion 200a′ may be utilized to non-removably secure the cannula carrier 100′ to the hub 14′, and, the second fastening portion 200b′ may be utilized to non-removably secure the cannula 12′ to the distal end 102_{D}′ of the cannula carrier 100′ as similarly described above with respect to the hypodermic interface assembly 10. Although the distal surface of the collar 200c′ of the cannula 12′ may be disposed adjacent the first proximal end surface portion 104a′ of the proximal end surface 104′ of the substantially tube-shaped body 102′ of the cannula carrier 100′, the distal surface of the collar 200c′ may be said to be connected to the first proximal end surface portion 104a′ of the proximal end surface 104′ of the substantially tube-shaped body 102′ of the cannula carrier 100′ in a non-mated, non-adhesive configuration whereby an axial pulling force applied to the cannula 12′ would result in the distal surface of the collar 200c′ being pulled adjacent the proximal end surface 104′ of the substantially tube-shaped body 102′ of the cannula carrier 100′ in an interference-fit connection.

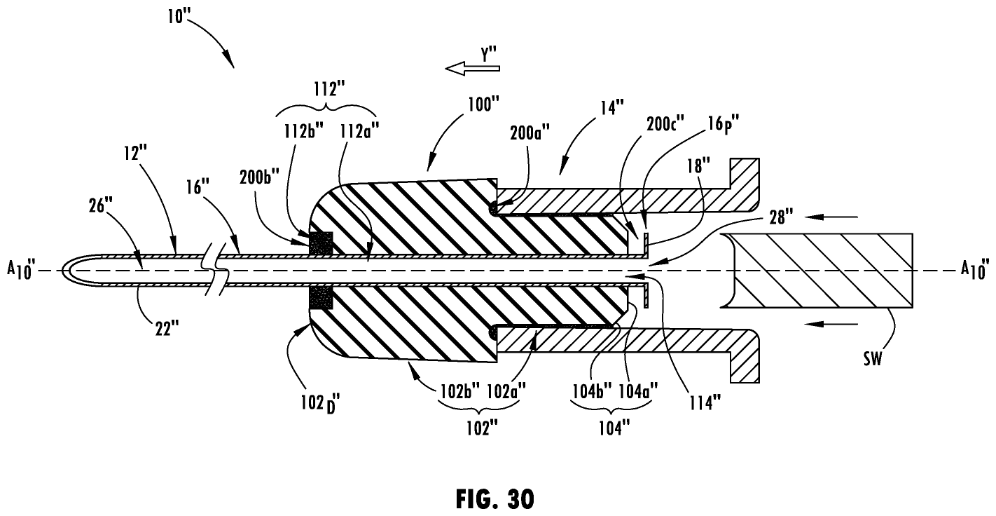

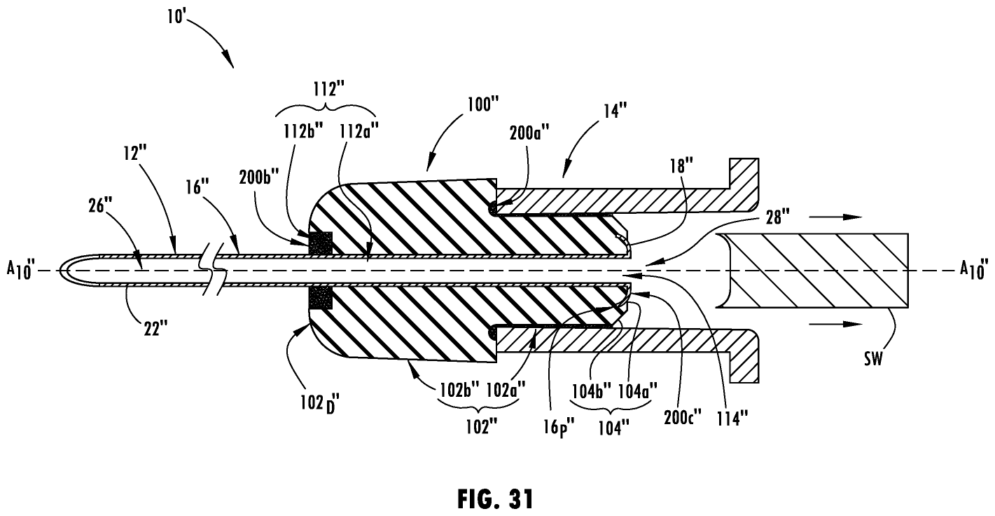

Referring to FIGS. 29-31, another hypodermic interface assembly is shown generally at 10″. The hypodermic interface assembly 10″ is substantially similar to the design of the hypodermic interface assembly 10′ with the exception that after the distal surface of the collar 200c″ of the hypodermic interface assembly 10″ is disposed adjacent the first proximal end surface portion 104a″ of the proximal end surface 104″ of the substantially tube-shaped body 102″ of the cannula carrier 100″, the distal surface of the collar 200c″ is deformed or swaged (by a swaging device SW as seen at FIGS. 30 and 31) such that the swaged distal surface of the collar 200c″ forms a male portion that extends into a corresponding female recess of the first proximal end surface portion 104a″ of the proximal end surface 104″ of the substantially tube-shaped body 102″ of the cannula carrier 100″ that is formed in response to the swaging of the of the collar 200c″ by the swaging device SW.

In the exemplary configuration of the hypodermic interface assembly 10″, the first fastening portion 200a″ may be utilized to non-removably secure the cannula carrier 100″ to the hub 14″, and, the second fastening portion 200b″ may be utilized to non-removably secure the cannula 12″ to the distal end 102_{D}″ of the cannula carrier 100″ as similarly described above with respect to the hypodermic interface assembly 10 and the hypodermic interface assembly 10′. The deformed or swaged distal surface of the collar 200c″ differs from the (non-deformed or non-swaged) collar 200c′ in that the deformed or swaged collar 200c″ may be said to be connected to the first proximal end surface portion 104a″ of the proximal end surface 104″ of the substantially tube-shaped body 102″ of the cannula carrier 100″ in a mechanically-mated, non-adhesive configuration whereby an axial pulling force applied to the cannula 12″ would result in the deformed or swaged distal surface of the collar 200c″ being pulled into the formed female recess of the proximal end surface 104″ of the substantially tube-shaped body 102′ of the cannula carrier 100″.

Another exemplary hypodermic interface assembly (not shown) may be substantially similar to the design of the hypodermic interface assembly 10″ in that it includes a deformed or swaged distal surface of the collar 200c″; however, it may not include a first fastening portion (e.g., first fastening portion 200a″) that would otherwise non-removably secure the cannula carrier (e.g., cannula carrier 100″) to the hub (e.g., hub 14″) or a second fastening portion (e.g., second fastening portion 200b″) that would otherwise non-removably secure the cannula (e.g., the cannula 12″) to the distal end (see, e.g., distal end 102_{D}″) of the cannula carrier.

Furthermore, another exemplary hypodermic interface assembly (not shown) may be arranged as follows. Rather than including a first fastening portion (e.g., first fastening portion 200a″) for adhesively securing the cannula carrier (e.g., cannula carrier 100″) to the hub (e.g., hub 14″), the first outer side surface portion (e.g., first outer side surface portion 108a″) of the outer side surface (e.g., outer side surface 108″) of the substantially tube-shaped body (e.g., substantially tube-shaped body 102″) of the cannula carrier (e.g., cannula carrier 100″) may be threadingly-secured in a mechanically-mated, non-adhesive configuration to the inner surface (e.g., inner surface 42″) of the substantially tube-shaped body (e.g., substantially tube-shaped body 34″) of the hub.

Yet another exemplary hypodermic interface assembly (not shown) may be arranged as follows. Rather than including a second fastening portion (e.g., second fastening portions 200b/200b′/200b″) for adhesively securing the cannula (e.g., cannulas 12, 12′, 12″) to the distal end (e.g., distal ends 102_{D}, 102_{D}′, 102_{D}″) of the cannula carrier (e.g., cannula carriers, 100, 100′, 100″), the material defining the cannula carrier may fill a region of the distal ends of the cannula carrier that would otherwise define a second passage portion (e.g., second passage portions 112b, 112b′) of the passage (e.g., passage 112, 112′, 112″); accordingly, the cannula may be said to be friction-fittingly-secured (i.e., in a non-mated, non-adhesive configuration) to the distal end of the cannula carrier.

Referring now to FIGS. 32 and 33A-33F, a methodology for utilizing any of the hypodermic interface assemblies 10, 10′, 10″ is shown. Although FIGS. 32 and 33A-33F show a methodology for utilizing any of the hypodermic interface assemblies 10, 10′, 10″, the hypodermic interface assembly 10 is represented at FIGS. 32 and 33A-33F; accordingly, although components of the hypodermic interface assembly 10 (such as, for example, the cannula 12 and the hub 14) are represented at FIGS. 32 and 33A-33F, any of the components of the other hypodermic interface assemblies 10′, 10″ may operate and function in a substantially similar manner.

As described above, the design of any hypodermic interface assembly 10 promotes predictable and controlled bending or flexing of one or more portions of the cannula carrier 100 instead of the cannula 12 relative to the hub 14 (see, e.g., FIG. 33D). In some instances, predictable and controlled bending or flexing of one or more portions of the cannula carrier 100 instead of the cannula 12 relative to the hub 14 may occur after the cannula 12 pierces the subject S (see, e.g., FIGS. 33A-33B). The subject S may be, for example, animalia, such as a human or non-human (i.e., an animal such as, for example, pig or swine). In other examples, the subject S may be an inanimate object. The predicable and controlled bending or flexing of one or more portions of the cannula carrier 100 instead of the cannula 12 relative to the hub 14 mitigates separation of the cannula 12 from the hub 14, which may otherwise result in the cannula 12 being broken-off and subsequently lost within the flesh of the animalia.

Referring to FIG. 32, the hypodermic interface assembly 10 is shown connected to an injecting device I, such as, for example, an injection gun. The hypodermic interface assembly 10 may be connected to a barrel portion I_{B} of the injection gun I by arranging, for example, the first radially-outward projection or ear 56 and the second radially-outward projection or ear 58 extending from the of the barrel-engaging portion 50 that extends from the outer surface 40 of the substantially tube-shaped body 34 of the hub 14 in corresponding recesses (not shown) formed by the barrel portion I_{B} of the injection gun I and then, for example, quarter-turn locking the hypodermic interface assembly 10 for removably-securing the first radially-outward projection or ear 56 and the second radially-outward projection or ear 58 extending from the of the barrel-engaging portion 50 to the barrel portion I_{B} of the injection gun I.

The injection gun I may include a fluid container C that contains a fluid F (see also, e.g., FIG. 33C). The fluid F may be metered from: (1) the container C; (2) through the injection gun I; (3) into the hypodermic interface assembly 10; and (4) out of the hypodermic interface assembly 10 and into the flesh of the subject S. The injection gun I may be actuated when a user U presses, for example, an actuator IA such as, for example, a trigger in order to cause movement of the fluid F as described above. The injection gun I may be powered in any desirable manner such as, for example: battery powered; air powered; manually powered; or a combination thereof.

Referring to FIG. 33A, the user may grasp the injection gun I and position the sharp piercing tip 32 formed by the distal end surface 20 of the tube-shaped body 16 of the cannula 12 near the outer surface S_{S} of the subject S, which may define the skin or hide of the subject S. Referring to FIGS. 33A-33B, the user U may impart an axial force according to the direction of the arrow XA to the injection gun I along the central axis A₁₀-A₁₀ extending through the hypodermic interface assembly 10 such that the sharp piercing tip 32 formed by the distal end surface 20 of the tube-shaped body 16 of the cannula 12 axially pierces the outer surface S_{S} of the subject S.

Referring to FIGS. 19E and 33C, after the outer surface S_{S} of the subject S has been axially pierced by the cannula 12, the user U may optionally actuate the actuator IA in order to cause movement of the fluid F from: (1) the container C; (2) through the injection gun I; (3) into the hypodermic interface assembly 10; and (4) out of the hypodermic interface assembly 10 and into the flesh of the subject S. In an example, the fluid F may firstly enter the hypodermic interface assembly 10 from the injection gun I at the passage 44 formed by the substantially tube-shaped body 34 of the hub 14 by way of the proximal opening 46 formed by the proximal end surface 36 of the substantially tube-shaped body 34 of the hub 14; and then secondly enter the passage 26 extending through the tube-shaped body 16 of the cannula 12 by way of the proximal opening 28 formed by the proximal end surface 18 of the body 16 of the cannula 12. Thereafter, the fluid F may exit the passage 26 extending through the tube-shaped body 16 of the cannula 12 by way of the distal opening 30 formed by the distal end surface 20 of the body 16 of the cannula 12.

The fluid F may be any desirable composition that is intended to be delivered to the animalia S. In some instances, the fluid F may be a medicament, a pharmaceutical, a vaccine, an anesthetic, or the like. Accordingly, the fluid F may not include any type of fluid that is not intended to be injected into animalia S. Although the hypodermic interface assembly 10 also may be utilized for injecting fluid F into animalia S, the hypodermic interface assembly 10 may be utilized for removing fluid F (e.g., blood) from animalia S. Therefore, it will be appreciated that the hypodermic interface assembly 10 may deliver or receive fluid F.

Referring to FIG. 33D, after the outer surface S_{S} of the subject S has been axially pierced by the cannula 12, the subject S may experience discomfort as a result of pain arising from the outer surface S_{S} being pierced by the sharp piercing tip 32 formed by the distal end surface 20 of the tube-shaped body 16 of the cannula 12. Accordingly, if the user U is sufficiently grasping the injection gun I, any movement of the subject S may result in the cannula 12 being subjected to one or more radial forces X_{R} relative to the central axis A₁₀-A₁₀ (extending through the hypodermic interface assembly 10) that may cause the cannula carrier 100 to bend or warp such that the entirety of the cannula 12 is axially shifted. This shift may thereby result in the central axis A₁₂-A₁₂ (extending through the axial center of the tube-shaped body 16 of the cannula 12) to not be coincident with the central axis A₁₀-A₁₀.

However, because the hub 14 may be formed from a non-flexible or substantially rigid material (e.g., metal), and, because the cannula carrier 100 may be formed from a flexible or substantially non-rigid material (e.g., plastic), at least a portion of the cannula carrier 100 may be permitted to flex or bend (as a result of radial movement of the cannula 12) relative to the hub 14 such that at least a portion of the central axis A₁₂-A₁₂ extending through the axial center of the tube-shaped body 16 of the cannula 12 is not coincident with the central axis A₁₀-A₁₀ extending through the hypodermic interface assembly 10. In an example, as seen at FIGS. 19E′, 19E″ and 33D, a portion (see, e.g., A₁₀₀′-A₁₀₀′) of the central axis A₁₀₀-A₁₀₀ of the cannula carrier 100 is permitted to deviate from the central axis A₁₀-A₁₀ extending through the hypodermic interface assembly 10 that may be entirely or partially coaxially aligned with central axes the other components of the hypodermic interface assembly 10. Accordingly, when, for example, the radial force X_{R} is applied to a cannula 12 that is connected to the cannula carrier 100, the head portion 102b of the substantially tube-shaped body 102 of the cannula carrier 100 may radially flex or bend away from the central axis A₁₀₀-A₁₀₀ of the cannula carrier 100 relative to the neck portion 102a of the substantially tube-shaped body 102 of the cannula carrier 100, which may be secured to the hub 14, and, as a result the neck portion 102a of the substantially tube-shaped body 102 of the cannula carrier 100 may not flex or bend away from the central axis A₁₀₀-A₁₀₀ of the cannula carrier 100. Accordingly, the head portion 102b of the substantially tube-shaped body 102 of the cannula carrier 100 may define a deformable portion of the substantially tube-shaped body 102 of the cannula carrier 100 that relieves radial stresses imparted to the cannula 12 arising from movement of the subject S during the probing or injecting procedure associated with the sharp piercing tip 32 formed by the distal end surface 20 of the tube-shaped body 16 of the cannula 12 piercing the skin S_{S} or hide of the subject S. Accordingly, a location of stress concentration applied to the cannula 12 arising from the radial force X_{R} imparted to the cannula 12 may be axially moved away from the hub 14 and closer to the subject S, which may be defined for example, at a location near the distal end 102_{D} of the substantially tube-shaped body 102 of the cannula carrier 100, which may be a distal-most portion of the head portion 102b of the substantially tube-shaped body 102 of the cannula carrier 100.

Referring to FIG. 33E, the user U, may withdraw the cannula 12 from the skin S_{S} or hide of the subject S. As seen at FIG. 33E, the cannula 12 does not break or separate from the hub 14 of the hypodermic interface assembly 10. As seen at FIG. 33F, the material defining the cannula carrier 100 may reflex or return to its pre-deformed state, and, as a result, may similarly move the cannula 12 for coaxially realigning its central axis A₁₂-A₁₂ with, for example, the central axis A₁₄-A₁₄ extending through the axial center of the hub 14. Therefore, as a result of the design of the hypodermic interface assembly 10, the cannula 12 is less likely to be broken-off and subsequently lost within the flesh of the subject S.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” “directly coupled to,” or “directly joins” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

What is claimed is:

1. A hypodermic interface assembly comprising:
a hub including: a proximal end having a proximal end surface; a distal end having a distal end surface; an outer side surface extending between and connecting the proximal end surface to the distal end surface; and an inner surface extending between and connecting the proximal end surface to the distal end surface, wherein the inner surface defines a passage extending through the hub from the proximal end surface to the distal end surface;
a cannula carrier friction-fit connected to the hub, wherein the cannula carrier includes an outer side surface including a first outer side surface portion, a second outer side surface portion, and a third outer side surface portion; and
a cannula connected to the cannula carrier, wherein the cannula extends through a passage formed by the cannula carrier.

2. The hypodermic interface assembly of claim 1, wherein the cannula carrier further includes:
a neck portion; and
a head portion extending from the neck portion, wherein the neck portion is defined by the first outer side surface portion, wherein the head portion is defined by the second outer side surface portion and the third outer side surface portion.

3. The hypodermic interface assembly of claim 2, wherein the neck portion is disposed within the passage formed by the hub.

4. The hypodermic interface assembly of claim 3, wherein the first outer side surface portion of the neck portion is arranged proximate an inner surface that defines the passage extending through the hub.

5. The hypodermic interface assembly of claim 1, wherein the third outer side surface portion of the head portion is disposed proximate the distal end surface of the distal end of the hub.

6. The hypodermic interface assembly of claim 2, wherein the first outer side surface portion is defined by a neck portion geometry, wherein the second outer side surface portion is defined by a head portion geometry that is greater than the neck portion geometry.

7. The hypodermic interface assembly of claim 1, wherein the third outer side surface portion is a proximal-side-facing surface that joins the first outer side surface portion to the second outer side surface portion.

8. A hypodermic interface assembly comprising:
a hub including: a proximal end having a proximal end surface; a distal end having a distal end surface; an outer side surface extending between and connecting the proximal end surface to the distal end surface; and an inner surface extending between and connecting the proximal end surface to the distal end surface, wherein the inner surface defines a passage extending through the hub from the proximal end surface to the distal end surface;
a cannula carrier connected to the hub, wherein the cannula carrier includes an outer side surface including a first outer side surface portion, a second outer side surface portion, and a third outer side surface portion;
a cannula connected to the cannula carrier, wherein the cannula extends through a passage formed by the cannula carrier; and
one or more fastening portions that non-removably-secures one or both of:
the cannula carrier to the hub; and
the cannula to the cannula carrier.

9. The hypodermic interface assembly of claim 8, wherein the one or more fastening portions includes:
one fastening portion that non-removably-secures the cannula carrier to the hub.

10. The hypodermic interface assembly of claim 8, wherein the one or more fastening portions includes:
one fastening portion that non-removably-secures the cannula to the cannula carrier.

11. The hypodermic interface assembly of claim 8, wherein the one or more fastening portions includes:
a first fastening portion that non-removably-secures a first portion of the cannula to a proximal end of the cannula carrier; and
a second fastening portion that non-removably-secures a second portion of the cannula to a proximal end of the cannula carrier.

12. The hypodermic interface assembly of claim 8, wherein the one or more fastening portions includes:
a first fastening portion that non-removably-secures the cannula carrier to the hub;
a second fastening portion that non-removably-secures a first portion of the cannula to a proximal end of the cannula carrier; and
a third fastening portion that non-removably-secures a second portion of the cannula to a proximal end of the cannula carrier.

13. The hypodermic interface assembly of claim 8, wherein the cannula carrier further includes:
a neck portion; and
a head portion extending from the neck portion, wherein the neck portion is defined by the first outer side surface portion, wherein the head portion is defined by the second outer side surface portion and the third outer side surface portion.

14. The hypodermic interface assembly of claim 13, wherein the neck portion is disposed within the passage formed by the hub.

15. The hypodermic interface assembly of claim 14, wherein the first outer side surface portion of the neck portion is arranged proximate an inner surface that defines the passage extending through the hub.

16. The hypodermic interface assembly of claim 8, wherein the third outer side surface portion of the head portion is disposed proximate the distal end surface of the distal end of the hub.

17. The hypodermic interface assembly of claim 13, wherein the first outer side surface portion is defined by a neck portion geometry, wherein the second outer side surface portion is defined by a head portion geometry that is greater than the neck portion geometry.

18. The hypodermic interface assembly of claim 8, wherein the third outer side surface portion is a proximal-side-facing surface that joins the first outer side surface portion to the second outer side surface portion.

19. A hypodermic interface assembly comprising:
a hub including: a proximal end having a proximal end surface; a distal end having a distal end surface; an outer side surface extending between and connecting the proximal end surface to the distal end surface; and an inner surface extending between and connecting the proximal end surface to the distal end surface, wherein the inner surface defines a passage extending through the hub from the proximal end surface to the distal end surface;
a cannula carrier connected to the hub, wherein the cannula carrier includes an outer side surface including a first outer side surface portion, a second outer side surface portion, and a third outer side surface portion; and
a cannula connected to the cannula carrier, wherein the cannula extends through a passage formed by the cannula carrier,
wherein the cannula carrier is formed from a material having a hardness that permits at least a portion of the cannula carrier to deform and deviate from:
an at-rest state axis extending through the cannula carrier and then to
a flexed axis extending through the portion of the cannula carrier upon application of a force imparted to the cannula that is not coaxial with the at-rest state axis extending through the cannula carrier and then back to
the at-rest state axis extending through the cannula carrier,
whereby a location of stress concentration applied to the cannula arising from the force imparted to the cannula is axially shifted from the hub toward a distal end of the cannula carrier for reinforcing a structural integrity of the cannula.

20. A method comprising:
providing a hub; and
providing a cannula carrier friction-fit connected to the hub, wherein the cannula carrier includes an outer side surface including a first outer side surface portion, a second outer side surface portion, and a third outer side surface portion
non-separably joining a cannula to the cannula carrier, wherein the cannula extends through a passage formed by the cannula carrier.

21. The method of claim 20 further comprising:
separably joining the hub to an injection gun; and
inserting the cannula into the flesh of a subject.

22. The method of claim 20 further comprising:
subjecting one or both of the cannula and the hub to one or more radial forces relative to a central axis extending through the cannula and the hub,
whereby a central axis extending through the cannula carrier is permitted to deviate from the central axis extending through the hub while the hub remains separably joined to the injection gun and the cannula is removably disposed within the flesh of the subject.
